SEQUENCE LISTING (16 sequences)

SEQ ID NO: 1 - DNA, 1070 bp, synthetic construct
  misc_feature 1..1070: note = expression construct
  source 1..1070: mol_type = other DNA
gccctgcaggtaaaaaaaggattgtgacttctccagcaacactgaaagttctcaacgcctacttggataaccccactccaaccctggaggaggcaattgaggtgttcaccccgctgaccgtgggtgaatacgatgacgtgcacatcgcagcgctgcttgccaccatccgtactcgcggtgagcagttcgctgatattgccggcgctgccaaggcgttcctcgcggcggctcgtccgttcccgattactggcgcaggtttgctagattccgctggtactggtggcgacggtgccaacaccatcaacatcaccaccggcgcatccctgatcgcagcatccggtggagtgaagctggttaagcacggcaaccgttcggtgagctccaagtccggctccgccgatgtgctggaagcgctgaatattcctttgggccttgatgtggatcgtgctgtgaagtggttcgaagcgtccaacttcaccttcctgttcgcacctgcgtacaaccctgcgattgcgcatgtgcagccggttcgccaggcgctgaaattccccaccatcttcaacacgcttggaccattgctgtccccggcgcgcccggagcgtcagatcatgggcgtggccaatgccaatcatggacagctcatcgccgaggtcttccgcgagttgggccgtacacgcgcgcttgttgtgcatggcgcaggcaccgatgagatcgcagtccacggcaccaccttggtgtgggagcttaaagaagacggcaccatcgagcattacaccatcgagcctgaggaccttggccttggccgctacacccttgaggatctcgtaggtggcctcggcactgagaacgccgaagctatgcgcgctactttcgcgggcaccggccctgatgcacaccgtgatgcgttggctgcgtccgcaggtgcgatgttctacctcaacggcgatgtcgactccttgaaagatggtgcacaaaaggcgctttccttgcttgccgacggcaccacccaggcatggttggccaagcacgaagagatcgattactcagaaaaggagtcttccaatgactag

SEQ ID NO: 2 - DNA, 1070 bp, synthetic construct
  misc_feature 1..1070: note = expression construct
  source 1..1070: mol_type = other DNA
gccctgcaggtaaaaaaaggattttgacttctccagcaacactgaaagttctcaacgcctacttggataaccccactccaaccctggaggaggcaattgaggtgttcaccccgctgaccgtgggtgaatacgatgacgtgcacatcgcagcgctgcttgccaccatccgtactcgcggtgagcagttcgctgatattgccggcgctgccaaggcgttcctcgcggcggctcgtccgttcccgattactggcgcaggtttgctagattccgctggtactggtggcgacggtgccaacaccatcaacatcaccaccggcgcatccctgatcgcagcatccggtggagtgaagctggttaagcacggcaaccgttcggtgagctccaagtccggctccgccgatgtgctggaagcgctgaatattcctttgggccttgatgtggatcgtgctgtgaagtggttcgaagcgtccaacttcaccttcctgttcgcacctgcgtacaaccctgcgattgcgcatgtgcagccggttcgccaggcgctgaaattccccaccatcttcaacacgcttggaccattgctgtccccggcgcgcccggagcgtcagatcatgggcgtggccaatgccaatcatggacagctcatcgccgaggtcttccgcgagttgggccgtacacgcgcgcttgttgtgcatggcgcaggcaccgatgagatcgcagtccacggcaccaccttggtgtgggagcttaaagaagacggcaccatcgagcattacaccatcgagcctgaggaccttggccttggccgctacacccttgaggatctcgtaggtggcctcggcactgagaacgccgaagctatgcgcgctactttcgcgggcaccggccctgatgcacaccgtgatgcgttggctgcgtccgcaggtgcgatgttctacctcaacggcgatgtcgactccttgaaagatggtgcacaaaaggcgctttccttgcttgccgacggcaccacccaggcatggttggccaagcacgaagagatcgattactcagaaaaggagtcttccaatgactag

SEQ ID NO: 3 - protein, 348 aa, Corynebacterium glutamicum
MTSPATLKVLNAYLDNPTPTLEEAIEVFTPLTVGEYDDVHIAALLATIRTRGEQFADIAGAAKAFLAAARPFPITGAGLLDSAGTGGDGANTINITTGASLIAASGGVKLVKHGNRSVSSKSGSADVLEALNIPLGLDVDRAVKWFEASNFTFLFAPAYNPAIAHVQPVRQALKFPTIFNTLGPLLSPARPERQIMGVANANHGQLIAEVFRELGRTRALVVHGAGTDEIAVHGTTLVWELKEDGTIEHYTIEPEDLGLGRYTLEDLVGGLGTENAEAMRATFAGTGPDAHRDALAASAGAMFYLNGDVDSLKDGAQKALSLLADGTTQAWLAKHEEIDYSEKESSND

SEQ ID NO: 4 - protein, 919 aa, Corynebacterium glutamicum
MTDFLRDDIRFLGQILGEVIAEQEGQEVYELVEQARLTSFDIAKGNAEMDSLVQVFDGITPAKATPIARAFSHFALLANLAEDLYDEELREQALDAGDTPPDSTLDATWLKLNEGNVGAEAVADVLRNAEVAPVLTAHPTETRRRTVFDAQKWITTHMRERHALQSAEPTARTQSKLDEIEKNIRRRITILWQTALIRVARPRIEDEIEVGLRYYKLSLLEEIPRINRDVAVELRERFGEGVPLKPVVKPGSWIGGDHDGNPYVTAETVEYSTHRAAETVLKYYARQLHSLEHELSLSDRMNKVTPQLLALADAGHNDVPSRVDEPYRRAVHGVRGRILATTAELIGEDAVEGVWFKVFTPYASPEEFLNDALTIDHSLRESKDVLIADDRLSVLISAIESFGFNLYALDLRQNSESYEDVLTELFERAQVTANYRELSEAEKLEVLLKELRSPRPLIPHGSDEYSEVTDRELGIFRTASEAVKKFGPRMVPHCIISMASSVTDVLEPMVLLKEFGLIAANGDNPRGTVDVIPLFETIEDLQAGAGILDELWKIDLYRNYLLQRDNVQEVMLGYSDSNKDGGYFSANWALYDAELQLVELCRSAGVKLRLFHGRGGTVGRGGGPSYDAILAQPRGAVQGSVRITEQGEIISAKYGNPETARRNLEALVSATLEASLLDVSELTDHQRAYDIMSEISELSLKKYASLVHEDQGFIDYFTQSTPLQEIGSLNIGSRPSSRKQTSSVEDLRAIPWVLSWSQSRVMLPGWFGVGTALEQWIGEGEQATQRIAELQTLNESWPFFTSVLDNMAQVMSKAELRLAKLYADLIPDTEVAERVYSVIREEYFLTKKMFCVITGSDDLLDDNPLLARSVQRRYPYLLPLNVIQVEMMRRYRKGDQSEQVSRNIQLTMNGLSTALRNSG

SEQ ID NO: 5 - DNA, 2760 bp, Corynebacterium glutamicum
  source 1..2760: mol_type = other DNA
atgactgattttttacgcgatgacatcaggttcctcggtcaaatcctcggtgaggtaattgcggaacaagaaggccaggaggtttatgaactggtcgaacaagcgcgcctgacttcttttgatatcgccaagggcaacgccgaaatggatagcctggttcaggttttcgacggcattactccagccaaggcaacaccgattgctcgcgcattttcccacttcgctctgctggctaacctggcggaagacctctacgatgaagagcttcgtgaacaggctctcgatgcaggcgacacccctccggacagcactcttgatgccacctggctgaaactcaatgagggcaatgttggcgcagaagctgtggccgatgtgctgcgcaatgctgaggtggcgccggttctgactgcgcacccaactgagactcgccgccgcactgtttttgatgcgcaaaagtggatcaccacccacatgcgtgaacgccacgctttgcagtctgcggagcctaccgctcgtacgcaaagcaagttggatgagatcgagaagaacatccgccgtcgcatcaccattttgtggcagaccgcgttgattcgtgtggcccgcccacgtatcgaggacgagatcgaagtagggctgcgctactacaagctgagccttttggaagagattccacgtatcaaccgtgatgtggctgttgagcttcgtgagcgtttcggcgagggtgttcctttgaagcccgtggtcaagccaggttcctggattggtggagaccacgacggtaacccttatgtcaccgcggaaacagttgagtattccactcaccgcgctgcggaaaccgtgctcaagtactatgcacgccagctgcattccctcgagcatgagctcagcctgtcggaccgcatgaataaggtcaccccgcagctgcttgcgctggcagatgcagggcacaacgacgtgccaagccgcgtggatgagccttatcgacgcgccgtccatggcgttcgcggacgtatcctcgcgacgacggccgagctgatcggcgaggacgccgttgagggcgtgtggttcaaggtctttactccatacgcatctccggaagaattcttaaacgatgcgttgaccattgatcattctctgcgtgaatccaaggacgttctcattgccgatgatcgtttgtctgtgctgatttctgccatcgagagctttggattcaacctttacgcactggatctgcgccaaaactccgaaagctacgaggacgtcctcaccgagcttttcgaacgcgcccaagtcaccgcaaactaccgcgagctgtctgaagcagagaagcttgaggtgctgctgaaggaactgcgcagccctcgtccgctgatcccgcacggttcagatgaatacagcgaggtcaccgaccgcgagctcggcatcttccgcaccgcgtcggaggctgttaagaaattcgggccacggatggtgcctcactgcatcatctccatggcatcatcggtcaccgatgtgctcgagccgatggtgttgctcaaggaattcggactcatcgcagccaacggcgacaacccacgcggcaccgtcgatgtcatcccactgttcgaaaccatcgaagatctccaggccggcgccggaatcctcgacgaactgtggaaaattgatctctaccgcaactacctcctgcagcgcgacaacgtccaggaagtcatgctcggttactccgattccaacaaggatggcggatatttctccgcaaactgggcgctttacgacgcggaactgcagctcgtcgaactatgccgatcagccggggtcaagcttcgcctgttccacggccgtggtggcaccgtcggccgcggtggcggaccttcctacgacgcgattcttgcccagcccaggggggctgtccaaggttccgtgcgcatcaccgagcagggcgagatcatctccgctaagtacggcaaccccgaaaccgcgcgccgaaacctcgaagccctggtctcagccacgcttgaggcatcgcttctcgacgtctccgaactcaccgatcaccaacgcgcgtacgacatcatgagtgagatctctgagctcagcttgaagaagtacgcctccttggtgcacgaggatcaaggcttcatcgattacttcacccagtccacgccgctgcaggagattggatccctcaacatcggatccaggccttcctcacgcaagcagacctcctcggtggaagatttgcgagccatcccatgggtgctcagctggtcacagtctcgtgtcatgctgccaggctggtttggtgtcggaaccgcattagagcagtggattggcgaaggggagcaggccacccaacgcattgccgagctgcaaacactcaatgagtcctggccatttttcacctcagtgttggataacatggctcaggtgatgtccaaggcagagctgcgtttggcaaagctctacgcagacctgatcccagatacggaagtagccgagcgagtctattccgtcatccgcgaggagtacttcctgaccaagaagatgttctgcgtaatcaccggctctgatgatctgcttgatgacaacccacttctcgcacgctctgtccagcgccgatacccctacctgcttccactcaacgtgatccaggtagagatgatgcgacgctaccgaaaaggcgaccaaagcgagcaagtgtcccgcaacattcagctgaccatgaacggtctttccactgcgctgcgcaactccggctag

SEQ ID NO: 6 - protein, 185 aa, Corynebacterium glutamicum
MNDQIHLDHQSDDTSECSCPIVVLVGLPGAGKSTIGRRLARALNTELVDSDELIERATGKACGAVFSELGEPAFRELEAIHVAEALKSSGVVSLGGGSVLTESTRELLKGQDVVWIDVPVEEGIRRTANERSRPVLQAADPAEHYRNLVKVRTPLYEEVATYRLRTNNRSPQQVVAAVLHHLEID

SEQ ID NO: 7 - protein, 174 aa, Escherichia coli
MTQPLFLIGPRGCGKTTVGMALADSLNRRFVDTDQWLQSQLNMTVAEIVEREEWAGFRARETAALEAVTAPSTVIATGGGIILTEFNRHFMQNNGIVVYLCAPVSVLVNRLQAAPEEDLRPTLTGKPLSEEVQEVLEERDALYREVAHIIIDATNEPSQVISEIRSALAQTINC

SEQ ID NO: 8 - DNA, 179 bp, synthetic construct
  misc_feature 1..179: note = promoter sequence
  source 1..179: mol_type = other DNA
cacagggtagctggtagtttgaaaatcaacgccgttgcccttaggattcagtaactggcacattttgtaatgcgctagatctgtgtgctcagtcttccaggctgcttatcacagtgaaagcaaaaccaattcgtggctgcgaaagtcgtagccaccacgaagtccaggaggacatacat

SEQ ID NO: 9 - protein, 430 aa, Escherichia coli
MVFVSDSSISLPIWDAPRARGPIVSDLAIPGSKSITNRALILAALASTPSTIIDVLRSRDTDLMTDGLRSLGITITEEAVDRYRVEPGQLSAGSVECGLAGTVMRFLPPVAAFADGPVHFDGDPQARVRPMTSILDALRSLGVEVDNNNLPFTVNAGEVPEGGVVEIDASGSSQFVSGLLLSAPRFKNGVTVKHVGGRLPSMPHIEMTVDMLRSAGIEIEESENQWVVHPGEILGRTWRIEPDLSNATPFLAAAAVTGGTIKINHWPIKTTQPGDAIRSILERMGCEVELVAQGEGYDLSVTGPVALKGIEIDMSDIGELTPTVAALAALASTESRLTGIAHLRGHETDRLAALTAEINKLGGKCTELKDGLLIEPASLHGGVWHSYADHRMATAGAIIGLAVDGVQVEDIKTTSKTFPGFENVWEEMVG

SEQ ID NO: 10 - protein, 410 aa, Corynebacterium glutamicum
MLGMLRWTTAGESHGQALIATVEHMPAGVPVTKDEVSYQLARRRLGYGRGARMKFEQDALTFLTGIRHGLTLGSPISIMIGNTEWDKWTTIMSSDALDMEDPDNVAAMSSGRGAKLTRPRPGHADYAGMLKYGFDDARNVLERSSARETAARVAAATVARSFLRETLGVEVLSHVISIGASEPYTGAEPTFADIQAIDDSPVRAFGKDAEESMIAEIEAAKKAGDTLGGIVEVIVEGLPIGLGSHISGEDRLDAQIAAALMGIQAIKGVEIGDGFEEARRRGSEAHDEVFLDDNGVYRNTNRAGGLEGGMTNGETLRVRAGMKPISTVPRALKTIDMENGKAATGIHQRSDVCAVPAAGVVAEAMVTLVLARAVLQKFGGDSLSETKSNIDTYLKNIEERMKFEGLEDGA

SEQ ID NO: 11 - protein, 350 aa, synthetic construct
  REGION 1..350: note = modified enzyme
MNYQNDDLRIKEIKELLPPVALLEKFPATENAANTVAHARKAIHKILKGNDDRLLVVIGPCSIHDPVAAKEYATRVLALREELKDELEIVMRVYFEKPRTTVGWKGLINDPHMDNSFQINDGLRIARKLLLDINDSGLPAAGEFLDMITPQYLADLMSWGAIGARTTESQVHRELASGLSCPVGFKNGTDGTIKVAIDAINAAGAPHCFLFVTKWGHSAIVNTSGNGDCHIILRGGKEPNYSAKHVAEVKEGLNKAGLPAQVMIDFSHANSSKQFKKQMDVCADVCQQIAGGEKAIIGVMVESHLVEGNQSLESGEPLAYGKSITDACIGWEDTDALLRQLANAVKARRG

SEQ ID NO: 12 - protein, 446 aa, Xanthomonas campestris
MSNTVFIGAKEYFPGIGKIGFEGRDSDNPLAFKVYDANKQVAGKTMAEHLRFAVAYWHSFCGNGADPFGPGTRAYPWDVGNTALARAEAKSDAAFEFFTKLGVPYYCFHDIDLAPDADDIGEYENNLKHMVGIAKQRQADTGVKLLWGTANLFSHPRYMNGASTNPDFNVVARAAVQVKAAIDATVELGGENYVFWGGREGYACLHNTQMKREQDNMARFLTLARDYGRAIGFTGNFLIEPKPMEPMKHQYDFDSATVIGFLRQHGLDQDFKLNIEANHATLSGHSFEHDLQVASDAGLLGSIDANRGNPQNGWDTDQFPTDLYDTVGAMLVVLRQGGLAPGGLNFDAKVRRESSDPQDLFLAHIGGMDAFARGLEVANALLTSSPLETWRAQRYASFDSGAGADFANGTSTLADLAKYAAGKGEPTQVSGRQEAYENLINQYLTR

SEQ ID NO: 13 - protein, 463 aa, synthetic construct
  REGION 1..463: note = modified enzyme
MSNTVFIGAKEYFPGIGKIGFEGRDSDNPLAFKVYDANKQVAGKTMAEHLRFAVAYWHSFCGNGADPFGPGTRAYPWDVGNTALARAEAKSDAAFEFFTKLGVPYYCFHDIDLAPDADDIGEYENNLKHMVGIAKQRQADTGVKLLWGTANLFSHPRYMNGASTNPDFNVVARAAVQVKAAIDATVELGGENYVFWGGREGYACLHNTQMKREQDNMARFLTLARDYGRAIGFTGNFLIEPKPMEPMKHQYDFDSATVIGFLRQHGLDQDFKLNIEANHATLSGHSFEHDLQVASDAGLLGSIDANRGNPQNGWDTDQFPTDLYDTVGAMLVVLRQGGLAPGGLNFDAKVRRESSDPQDLFLAHIGGMDAFARGLEVANALLTSSPLETWRAQRYASFDSGAGADFANGTSTLADLAKYAAGKGEPTQVSGRQEAYENLINQYLTRHRVAGSLKINAVALRIQ

SEQ ID NO: 14 - protein, 460 aa, Corynebacterium glutamicum
MALVLGIDSSTQSCKALLVDAATGQVIDEGRASHPSGSEVDPRAWIAALDQATEGLLERADAVSIAGQQHGMVALDENDEIVRPALLWNDTRSAQAALDLNEEIGGDQAAVDATGSVYVASLTATKMRWMRDHEPENAARTASVMLPHDFLTWHLMGRGRKVTDHGDASGTGYYSTRDRAWRTDLAALALGHEVELPELLAPNAIAGTTPGGVKVAAGTGDNAAAALGLDLQPGDVSVSIGTSGVAGMTVQHSVHDPSGLVTGFADATGAYFPLACTLNGAPVLEFGRRILGVEWEEFDALALAAQPGSGGVTLQPYLEGERTPNRPAARGVLAGLNCATTREDFARATVEGLLLALDDAVTALVEATGVPVQRIQLIGGGARSQAVREIAPEIFGHEIVVPEPAEYVALGAARQAAWALSGEATPPQWPTPGSDPHRAPKNTELSTRYAKLRAATQGWY

SEQ ID NO: 15 - DNA, 3302 bp, synthetic construct
  misc_feature 1..3302: note = expression construct
  source 1..3302: mol_type = other DNA
gatcccacagggtagctggtagtttgaaaatcaacgccgttgcccttaggattcagtaactggcacattttgtaatgcgctagatctgtgtgctcagtcttccaggctgcttatcacagtgaaagcaaaaccaattcgtggctgcgaaagtcgtagccaccacgaagtccaggaggacatacagtatgacacaacctctttttctgatcgggcctcggggctgtggtaaaacaacggtcggaatggcccttgccgattcgcttaaccgtcggtttgtcgataccgatcagtggttgcaatcacagctcaatatgacggtcgcggagatcgtcgaaagggaagagtgggcgggatttcgcgccagagaaacggcggcgctggaagcggtaactgcgccatccaccgttatcgctacaggcggcggcattattctgacggaatttaatcgtcacttcatgcaaaataacgggatcgtggtttatttgtgtgcgccagtatcagtcctggttaaccgactgcaagctgcaccggaagaagatttacggccaaccttaacgggaaaaccgctgagcgaagaagttcaggaagtgctggaagaacgcgatgcgctatatcgcgaagttgcgcatattatcatcgacgcaacaaacgaacccagccaggtgatttctgaaattcgcagcgccctggcacagacgatcaattgttgacaatctttagaggagacacaacatggtctttgtgtctgattcgtctatctctttgcccatttgggatgctccgcgcgctcgcggccccatagtctcggacctggctatccctggttccaagtcgatcaccaaccgcgccctcatcttggctgcgctcgcatcaactccatccaccatcattgatgtccttcgtagtcgtgataccgatctcatgactgatggtctacgcagcctcggaatcaccattactgaagaggcagtcgatcgctaccgcgttgagcccggacagttgtctgctggctccgttgagtgtggtcttgctggtacggtcatgcgctttttgcctcctgttgctgctttcgctgatggtcctgttcattttgatggcgatcctcaagctcgtgttcgtccgatgaccagcattttggatgcgctgcgttcgcttggtgtggaggtagacaacaacaatctgcctttcactgttaatgctggtgaggtccctgagggtggcgtggttgagattgatgcttccggctcatctcagtttgtttctggtcttttgctttcagcgcctcgttttaaaaatggcgtcaccgttaagcacgtcggtggtcgtctgccgagcatgccgcatattgagatgaccgtcgatatgcttcgttccgcaggcattgagatcgaagagtcagaaaatcagtgggttgttcatcctggtgagatcttgggtcggacctggcgcattgagccggatctttctaatgcgactccgttcctagctgccgctgcggtcactggtggaaccatcaagattaaccactggccaatcaaaactactcagcctggcgatgctattcgttcgattcttgagcgcatgggctgcgaagttgagctggttgctcagggtgaaggttacgatctgtcggtgactggtccggttgctctcaagggcattgagatcgatatgtccgatatcggtgagttgacccctaccgtggcggcgttggctgcgttggcgtcgacagagtctcgtttgaccggtattgctcatcttcgtggccatgagacggatcgtttggctgcgttgactgcggagatcaacaaacttggtggaaagtgcactgagcttaaggatggtctgttgattgagcctgcgtcgctgcacggtggtgtgtggcattcatatgctgatcaccgtatggctactgctggtgcgatcattggcctcgcggttgatggcgttcaggttgaagacattaagaccacttccaaaactttccctggttttgaaaatgtttgggaggagatggttggctaggcgaaagtcgtagccaccacgaagtccgaaaggaggcccttcagatgctaggcatgcttcgatggactacagcaggtgaatcccacggccaggcgcttatcgccacggttgaacacatgccagcaggcgtgcccgtgactaaagatgaggtctcgtatcaattggcgcgccgacgccttggatatggtcgcggcgctcgcatgaagtttgagcaagacgcgttgaccttcctcaccggcatccgccacggcctcactttgggtagccccatctcaatcatgatcggcaacactgagtgggataagtggaccaccatcatgtcctctgacgctttggacatggaagacccagataacgttgcggcgatgtcttcgggtcggggcgcaaaactgactcgtccgcgtccaggccacgctgattacgcaggcatgctcaagtacggattcgatgatgcccgcaacgtgctggagcgttcttcagcccgtgagacggcagcacgcgtggcagcagcaaccgttgcgcgttccttcctgcgtgaaaccttgggcgtggaagtgctttcccacgtaatttccattggtgcgtccgagccttacactggcgcggagccaacctttgcagatattcaagcaatcgatgattccccagttcgtgcattcggtaaagacgctgaagaatccatgatcgcggaaatcgaggccgcaaagaaagccggcgataccctcggtggcatcgtggaagtgattgttgaaggcctgcccatcggtttgggctcacacatttctggcgaagatcgcctcgatgcgcagatcgcagctgcactcatgggcattcaggccatcaagggcgtggaaatcggtgacggtttcgaagaagctcgtcgacgtggctccgaagcccacgatgaagtgttcctggatgacaacggcgtataccgcaacaccaaccgtgcaggtggcctcgaaggcggcatgaccaacggtgaaaccctgcgcgttcgtgctggcatgaagccaatttctactgtgcctcgcgccctgaaaaccattgatatggaaaacggcaaggcagcaaccggaatccaccagcgttccgacgtgtgcgctgttccagccgccggtgtcgttgcagaagcaatggtcaccctggttctcgcccgcgcagtcctgcagaaattcggcggtgactccctgagcgaaaccaagagcaacattgacacctacctcaaaaacattgaggaacgaatgaaattcgaaggtttagaggatggagcgtaa

SEQ ID NO: 16 - DNA, 3539 bp, synthetic construct
  misc_feature 1..3539: note = expression construct
  source 1..3539: mol_type = other DNA
cacagggtagctggtagtttgaaaatcaacgccgttgcccttaggattcagtaactggcacattttgtaatgcgctagatctgtgtgctcagtcttccaggctgcttatcacagtgaaagcaaaaccaattcgtggctgcgaaagtcgtagccaccacgaagtccaggaggacatacatgaaaggaggcccttcagatgtccaacaccgtgttcatcggcgcaaaggaatacttcccaggcatcggcaagatcggcttcgaaggccgcgattccgataacccactggcattcaaggtgtacgatgcaaacaagcaggtggcaggcaagaccatggcagaacacctgcgcttcgcagtggcatactggcactccttctgcggcaacggcgcagatccattcggcccaggcacccgcgcatacccatgggatgtgggcaacaccgcactggcacgcgcagaagcaaagtccgatgcagcattcgaattcttcaccaagctgggcgtgccatactactgcttccacgatatcgatctggcaccagatgcagatgatatcggcgaatacgaaaacaacctgaagcacatggtgggcatcgcaaagcagcgccaggcagataccggcgtgaagctgctgtggggcaccgcaaacctgttctcccacccacgctacatgaacggcgcatccaccaacccagatttcaacgtggtggcacgcgcagcagtgcaggtgaaggcagcaatcgatgcaaccgtggaactgggcggcgaaaactacgtgttctggggcggccgcgaaggctacgcatgcctgcacaacacccagatgaagcgcgaacaggataacatggcacgcttcctgaccctggcacgcgattacggccgcgcaatcggcttcaccggcaacttcctgatcgaaccaaagccaatggaaccaatgaagcaccagtacgatttcgattccgcaaccgtgatcggcttcctgcgccagcacggcctggatcaggatttcaagctgaacatcgaagcaaaccacgcaaccctgtccggccactccttcgaacacgatctgcaggtggcatccgatgcaggcctgctgggctccatcgatgcaaaccgcggcaacccacagaacggctgggataccgatcagttcccaaccgatctgtacgataccgtgggcgcaatgctggtggtgctgcgccagggcggcctggcaccaggcggcctgaacttcgatgcaaaggtgcgccgcgaatcctccgatccacaggatctgttcctggcacacatcggcggcatggatgcattcgcacgcggcctggaagtggcaaacgcactgctgacctcctccccactggaaacctggcgcgcacagcgctacgcatccttcgattccggcgcaggcgcagatttcgcaaacggcacctccaccctggcagatctggcaaagtacgcagcaggcaagggcgaaccaacccaggtgtccggccgccaggaagcatacgaaaacctgatcaaccagtacctgacccgccacagggtagctggtagtttgaaaatcaacgccgttgcccttaggattcagtaactggcacattttgtaatgcgctagatctgtgtgctcagtcttccaggctgcttatcacagtgaaagcaaaaccaattcgtggctgcgaaagtcgtagccaccacgaagtccaggaggacatacatgaaaggaggcccttcagatggctttggttcttggaatcgatagttccacccaatcctgcaaggctttgcttgtcgatgccgccaccggccaggttatcgacgaaggccgcgcgagtcacccgagcgggtcggaggtagatccacgtgcgtggatcgctgcgctggatcaagctaccgaggggttgttagaacgcgcggacgctgtatctattgcaggccagcagcacggcatggtggcgttggatgaaaacgatgaaatcgttcgcccggcgttgttatggaatgacactcgttctgcccaggctgcgttggatctcaatgaggagatcggcggcgatcaggctgcggtagatgccacgggaagtgtgtatgttgcttctttaactgccaccaaaatgcggtggatgcgtgatcatgaaccagaaaatgcagcgcgcacggcgtcggtgatgttgcctcatgatttcctcacctggcatttgatgggacgcggacgcaaagtcaccgaccatggtgatgcttctggaacgggctactacagcacgcgtgatcgtgcgtggcgcaccgatctagctgccttggcgctgggccatgaggtggaacttcctgaactcctggccccaaatgcgattgcgggaacaactccaggtggagtgaaagttgctgcaggcacgggagataatgctgcggctgcgcttggccttgatttgcagcctggtgatgtcagcgtgtcgatcggcacctctggcgttgccggcatgaccgttcaacatagcgtccacgatccatctggtttggtcactggtttcgccgatgccacgggtgcgtatttcccgctggcctgcacgcttaatggcgcaccggtgttggaattcggccgccgcattctgggcgtggaatgggaagagttcgatgcgcttgcactggctgctcaacccggttcaggtggcgtgacgctccagccttatttggagggcgagcgtacgccgaatcgtcccgcagcacgtggcgttttggctggactaaactgtgcaacgacccgcgaggactttgcccgagcaactgttgaaggcttgttgttggcattggatgatgctgtaacggcgctggttgaggccacgggagtgcccgttcagcgcatccagctcatcggtggcggcgcgcgttcacaggcggttcgtgagattgcccctgagattttcggccatgagattgtggttccagaacccgctgaatatgtggcgttgggtgcagctcgtcaggcggcatgggcgctgtcgggtgaggccacgccaccgcagtggccaactcccggttccgatccgcaccgcgcacctaaaaacactgagctgagcacgcgttatgcgaagctgcgtgctgcaacgcagggttggtactagggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactct

(19) United States
(12) Patent Application Publication
(10) Pub. No.: US 2026/0117268 A1
(43) Pub. Date: Apr. 30, 2026

(54) MIXTURES OF GLUCOSE AND XYLOSE FOR THE FERMENTATIVE PREPARATION OF ORTHO-AMINOBENZOIC ACID

(71) Applicant: Covestro Deutschland AG, Leverkusen (DE)

(72) Inventors: Anja Spaeth, Monschau (DE); Frederik Walter, Bielefeld (DE); Cedric Davoudi, Erkelenz (DE); Wolf Kloeckner, Köln (DE); Lena Schaffert, Köln (DE)

(21) Appl. No.: 18/718,517

(22) PCT Filed: Dec. 14, 2022
(86) PCT No.: PCT/EP2022/085917

§ 371 Date: Jun. 11, 2024

(30) Foreign Application Priority Data
Dec. 17, 2021 (EP) 21215759.8

(51) Int. Cl. C12P 13/00; C12N 1/20; C12N 9/10; C12N 9/12; C12N 9/88; C12N 9/92; C12R 1/15

(52) U.S. Cl. CPC C12P 13/005; C12N 1/20; C12N 9/1092; C12N 9/1205; C12N 9/88; C12N 9/92; C12R 2001/15; C12Y 204/02018; C12Y 205/01054; C12Y 207/01017; C12Y 207/01071; C12Y 402/03005; C12Y 503/01005

(57) ABSTRACT

The present invention relates to the preparation of ortho-aminobenzoic acid by means of microbial fermentation, wherein mixtures of glucose and xylose are used as fermentable substrates.

The present invention relates to the production of ortho-aminobenzoic acid by microbial fermentation, using mixtures of glucose and xylose as fermentable substrates.

The production of ortho-aminobenzoic acid (oAB) with the aid of a number of genetically modified microorganisms is known from the prior art, for example from Balderas-Hernandez et al. (2009) “Metabolic engineering for improving anthranilate synthesis from glucose in Escherichia coli”, WO 2015/124687 and WO 2017/102853.

All these publications described the use of glucose. Individual publications, for example WO 2017/102853, also describe the use of other carbon sources. However, the benefits of the combination of glucose and xylose have not been disclosed.

Labib et al. (2021) “Metabolic engineering for microbial production of protocatechuate with Corynebacterium glutamicum”, Biotechnology and Bioengineering, 118: 4414-4427, describe a method in which protocatechuic acid is produced by fermentation on the basis of glucose and xylose. This was done using a strain in which glucose was involved solely in constructive metabolism and maintenance metabolism, and product formation was based exclusively on xylose. But the carbon yield of the method described therein is low. Of every mole of carbon in the total sugar (glucose and xylose) consumed by the cells, only 0.12 mol went into the product produced therein.

WP 3 061 828 describes a method of producing amino acids, especially glutamic acid, by Corynebacterium. There was no description of the release of oAB by these strains, nor was this the aim.

Kogure et al. (2016) “Metabolic engineering of Corynebacterium glutamicum for shikimate overproduction by growth-arrested cell reaction” describes genetic alterations to C. glutamicum that led to accumulation of shikimic acid in the medium during the steady-state growth phase. Reaction pathways that led to the consumption of shikimic acid were interrupted, and so no oAB was formed.

In the study underlying the present patent application, it was found that, surprisingly, a combination of glucose and xylose in particular ratios leads to a particularly efficient conversion of the sugars to ortho-aminobenzoic acid (oAB). Carbon yields achieved here were one third higher than reported for a similar method of producing protocatechuic acid.

This invention is defined in detail in the claims and in the description hereinafter.

In a first embodiment, the present invention relates to a method comprising the step of culturing one or more microbial cells that are able to convert glucose and xylose to oAB in a culture medium containing a mixture of glucose and xylose having a glucose content between 5% by weight and 86% by weight and a xylose content between 95% by weight and 14% by weight, where the proportions of glucose and xylose add up to 100%, and where oAB is produced.

### oAB Production

The incubation of at least one microbial cell in the culture medium of the invention under suitable incubation conditions has the effect that this cell converts the sugars present in the culture medium, preferably the glucose and the xylose, more preferably the xylose, to oAB. In a preferred embodiment of the present invention, metabolic pathways in the microbial cell are separated such that xylose is used exclusively for formation of oAB, while growth takes place on glucose. Product formation from glucose as well is not ruled out here. In another preferred embodiment of the present invention, metabolic pathways in the microbial cell are separated such that glucose is used exclusively for formation of oAB, while growth takes place on xylose. Product formation from xylose as well is not ruled out here.

According to the invention, for each gram of glucose and xylose which is consumed by the at least one microbial cell during incubation, at least 0.09 gram of oAB is formed. This corresponds to a carbon yield of at least 0.138 mol of carbon in the form of oAB per mole of carbon consumed in glucose and xylose. Under particularly favorable conditions, these values may be at least 0.1 g of oAB per g of glucose and xylose consumed, which corresponds to a carbon yield of at least 0.153 mol of carbon in oAB per mole of carbon in glucose and xylose.

The term oAB refers to ortho-aminobenzoic acid, also known by the name “anthranilic acid”. Since this compound has both a carboxyl group having acidic properties and an amino group having basic properties, the charge of the molecule depends on pH. In the context of the present application, the term “ortho-aminobenzoic acid” refers to the molecule irrespective of whether it has a positive charge, a negative charge or even no net charge.

### Culture Medium

Culture media suitable for culturing microbial cells, especially Corynebacterium, are known from the prior art. Suitable culture media contain at least one buffer to regulate the pH, sources of inorganic nutrients that can be utilized by the microorganism used, in particular nitrogen, sulfur and phosphorus, and also the trace elements required by the organism used. Depending on the microorganism, the addition of vitamins and/or complex media constituents such as peptone or yeast extract may be useful. Particularly suitable culture media are described in the working examples included in this application.

The culture medium obligatorily contains glucose and xylose, where the glucose content is between 5% by weight and 86% by weight and the xylose content between 95% by weight and 14% by weight, and the proportions of glucose and xylose add up to 100%, as energy source and carbon source for the microorganism. This serves both for the construction of biomass and for the formation of oAB. This does not rule out the presence of other energy sources and carbon sources. But it is preferable that the proportion by mass of the sum total of xylose and glucose in the total amount of sugars present in the culture medium is at least 70% by weight, preferably at least 80% by weight and more preferably at least 90% by weight. In a preferred embodiment of the present invention, there is at least one sugar selected from the group consisting of galactose, sucrose, arabinose, cellobiose, maltose and fructose in the culture medium, while complying with the above-defined total proportions of glucose and xylose. Irrespective of the presence of any of the aforementioned sugars, the culture medium in this embodiment may also contain lactic acid and/or acetic acid.

In a particularly preferred embodiment of the present invention, the proportion by mass of the sum total of xylose and glucose in the total amount of all energy sources and carbon sources utilizable by the microorganism that are present in the culture medium is at least 70% by weight, preferably at least 80% by weight and more preferably at least 90% by weight. The terms “energy source” and “carbon source” are known to those skilled in the art. In the context of heterotrophic microorganisms, they refer to those organic carbon compounds from which the microorganism in question can either obtain the energy required for its metabolism or which it can utilize for construction of biomass.

The proportion of glucose in the mixture of glucose and xylose is preferably 5% by weight to 86% by weight, more preferably 8% by weight to 86% by weight, even more preferably 12% by weight to 86% by weight and most preferably 16% by weight to 86% by weight, where xylose contributes the proportion lacking from 100% by weight in each case.

With regard to yield, a distinction is made in accordance with the invention between two values: substrate yield and process yield. In the case of substrate yield, the amount of oAB produced is based on the amount of glucose and xylose consumed by the microbial cells during the culturing. Residual amounts of glucose and xylose that have been added to the culture medium but are still present in the culture medium at the end of the cultivation are not taken into account. This may be advantageous in method variants where the culture medium is reused for fermentation after the oAB has been separated off. In the case of process yield, the amount of oAB produced is based on the amount of glucose and xylose added to the culture medium during the culturing, regardless of whether these substrates have been fully converted or are still present as residual amounts in the culture medium. In simplified terms, amounts of glucose and xylose that have not been consumed by the microbial cells at the end of cultivation are recorded as losses in the process yield.

In order to achieve a maximum process yield, it has been found to be advantageous when the proportion of glucose in the mixture of glucose and xylose is 40% by weight to 86% by weight, preferably 50% by weight to 86% by weight, more preferably 60% by weight to 86% by weight and even more preferably 63% by weight to 86% by weight, where xylose contributes the proportion lacking from 100% by weight in each case.

### Culturing

The term “culturing” refers to the incubation of one or more microbial cells in the above-described culture medium under conditions that enable metabolic activity of the cells. In particular, this means the establishment and maintenance of a suitable pH, a suitable oxygen saturation, a suitable temperature and a suitable osmolality. The person skilled in the art may select culture conditions that are suitable for the microorganism in question based on their expertise and the generally available literature. Corynebacterium, in particular Corynebacterium glutamicum, is preferably cultured at a pH between 6 and 8, at a temperature between 25° C. and 40° C. and at an osmolality between 400 and 2600 mOsmol/kg. The oxygen saturation is preferably kept as close as possible to the maximum achievable under standard pressure and standard atmosphere.

The metabolic activity preferably consists in the production of oAB and/or the construction of biomass, more preferably in the production of oAB. A suitable parameter that allows continuous monitoring of metabolic activity for process control is also the rate of oxygen uptake. This may be carried out continuously, for example by oxygen electrodes, and provides measurement results without delay, which can be used to adjust the culture conditions.

The process of incubation of at least one microbial cell in an above-described culture medium under conditions that enable the formation of oAB by the microbial cells is also referred to hereinafter as “microbial fermentation”.

### Microbial Cell

The microbial cell is preferably a heterotrophic bacterial cell, more preferably a bacterium of the Corynebacterium genus and especially preferably Corynebacterium glutamicum.

In a preferred embodiment of the present invention, the microbial cell is a Corynebacterium, preferably Corynebacterium glutamicum.

It has been found that, surprisingly, a strain having such genetic modifications converts glucose and xylose to oAB with particularly high carbon yield. Consequently, the present application, in a further embodiment, relates to a strain of the Corynebacterium genus, preferably Corynebacterium glutamicum and more preferably Corynebacterium glutamicum ATCC13032, which differs from the wild type at least in the following features:
(i) reduced activity of anthranilate phosphoribosyltransferase compared to the respective wild type, but where residual activity must be present. The residual activity is preferably between 10% and 60%, more preferably between 20% and 50%, of the native activity in C. glutamicum ATCC13032. This is preferably achieved by reduced expression of the gene for anthranilate phosphoribosyltransferase (trpD) compared to the wild type, although expression is not completely suppressed. This is preferably effected by using a promoter sequence which has a lower transcription activity compared to the endogenous promoter sequence or by modifying the distance of the ribosome binding site to the start codon of the trpD gene or by altering the start codon itself. In a preferred embodiment of the present invention, the activity of anthranilate phosphoribosyltransferase is reduced by deletion or inactivation of the gene for endogenous anthranilate phosphoribosyltransferase (trpD) and the replacement of this gene by a gene for an anthranilate phosphoribosyltransferase having a modified ribosomal binding site and optionally a modified start codon as defined in SEQ ID NO. 1 or 2, preferably SEQ ID NO. 2. The amino acid sequence of the anthranilate phosphoribosyltransferase preferably corresponds to the endogenous anthranilate phosphoribosyltransferase, more preferably as defined by SEQ ID NO. 3 or a variant thereof.
(ii) elevated activity of shikimate kinase. This is preferably achieved by increased expression of a corresponding enzyme. In one embodiment of the present invention, the activity is increased by enhanced expression of the gene for the endogenous shikimate kinase as defined in SEQ ID NO. 6 or a variant thereof. In another preferred embodiment, this is achieved by expression of an exogenous shikimate kinase, preferably as defined in SEQ ID NO. 7 or a variant thereof. Enhanced expression of a gene can be achieved by any method known to those skilled in the art, in particular by introducing two or more copies of the corresponding gene into the microorganism or by using stronger promoters to express the endogenous enzyme. A particularly preferred promoter for expression of foreign genes or enhanced expression of endogenous genes is Ptuf as defined in SEQ ID NO. 8.
(iv) enhanced activity of 3-phosphoshikimate 1-carboxyvinyltransferase and chorismate synthase. Preferably, these enzymes have an amino acid sequence as defined in SEQ ID NO. 9 or a variant thereof and SEQ ID NO. 10 or a variant thereof. This is preferably effected by introducing additional copies of the genes coding for these enzymes into the microorganism. The Ptuf promoter is preferably used to control expression.
(iv) presence of a 3-deoxyarabinoheptulosanate-7-phosphate synthase (DAHP synthase) which is “feedback-resistant”, i.e. is not inhibited by its product or by any product formed from the product. Preference is given to an enzyme having the amino acid sequence defined in SEQ ID NO. 11 or a variant thereof.
(v) elevated activity of xylose isomerase and of xylulokinase. This elevated activity is preferably achieved by enhanced expression of both genes. This enhanced expression can be achieved by any method known to those skilled in the art, in particular by introducing two or more copies of the corresponding gene into the microorganism or by using stronger promoters to express the endogenous enzyme. Preference is given in accordance with the invention to elevated expression of enzymes having amino acid sequences as defined in SEQ ID NO. 12 or SEQ ID NO. 13/SEQ ID NO. 14, or variants thereof. Preference is given to a xylose isomerase as defined in SEQ ID NO. 12 or a variant thereof.

In a preferred embodiment of the present invention, the gene that encodes phosphoenolpyruvate carboxylase as defined in SEQ ID NO. 4 is deleted or inactivated. This may be effected in any manner familiar to those skilled in the art, preferably by deleting the gene or a partial sequence thereof, by introducing at least one stop codon or by deleting or inactivating the promoter sequence. Particular preference is given to the deletion of at least part of the protein-coding sequence of the gene (SEQ ID NO. 5).

With regard to anthranilate phosphoribosyltransferase (SEQ ID NO. 3), shikimate kinase (SEQ ID NO. 6 or 7), 3-phosphoshikimate 1-carboxyvinyltransferase (SEQ ID NO. 9), chorismate synthase (SEQ ID NO. 10), xylose isomerase (SEQ ID NO. 12) and xylulokinase (SEQ ID NO. 14), what is meant by “variant” is an enzyme which is obtained by adding, deleting or exchanging up to 10%, preferably up to 5%, of the amino acids present in the respective enzyme. The aforementioned modifications may in principle be executed continuously or discontinuously at any desired point in the enzyme. However, they are preferably executed solely at the N-terminus and/or at the C-terminus of the polypeptide. Amino acid substitutions are preferably conservative substitutions, i.e. those in which the modified amino acid has a residue with similar chemical properties to the amino acid present in the unaltered enzyme. Amino acids having basic residues are therefore more preferably exchanged for those with basic residues, amino acids having acidic residues for those with likewise acidic residues, amino acids having polar residues for those with polar residues, and amino acids having nonpolar residues for those with nonpolar residues. The specific enzyme activity of a variant is preferably at least 80% of the specific activity of the unmodified enzyme. Enzyme tests to verify the activity of the aforementioned enzymes can be found in the literature by those skilled in the art.

With regard to DAHP synthase (SEQ ID NO. 11), what is meant by “variant” is an enzyme obtained by adding, deleting or exchanging up to 5%, preferably up to 2%, of the amino acids present in the respective enzyme, with the proviso that positions 76 and 211 remain unaltered. It is also preferred that positions 10, 13, 147, 148, 150, 151, 179, 209, 211 and 212 additionally remain unaltered. In a more preferred embodiment of the present invention, positions 144, 175 and 215 are additionally also unaltered in the variant. In an even more preferred embodiment of the present invention, positions 92, 97, 165, 186 and 268 are also unaltered in addition to the aforementioned positions. The skilled person will appreciate that the aforementioned positions move accordingly if amino acids are deleted or inserted. The aforementioned modifications may in principle be executed continuously or discontinuously at any desired point in the enzyme. However, they are preferably executed solely at the N-terminus and/or at the C-terminus of the polypeptide. Amino acid substitutions are preferably conservative substitutions, i.e. those in which the modified amino acid has a residue with similar chemical properties to the amino acid present in the unaltered enzyme. Amino acids having basic residues are therefore more preferably exchanged for those with basic residues, amino acids having acidic residues for those with likewise acidic residues, amino acids having polar residues for those with polar residues, and amino acids having nonpolar residues for those with nonpolar residues. It is particularly preferred that the variant of DAHP synthase also possesses an appropriate enzyme activity. The specific enzyme activity of the variant is more preferably at least 80% of the specific activity of the unmodified DAHP synthase according to SEQ ID NO. 11.

In yet a further embodiment, the present invention relates to the use of a culture medium comprising a mixture of glucose and xylose having a glucose content between 5% by weight and 86% by weight and a xylose content between 95% by weight and 14% by weight, where the proportions of glucose and xylose add up to 100%, for production of oAB by a microbial fermentation.

Based on the above-defined genetic modifications, the cell is capable of releasing oAB into the culture medium and preferably also of enriching it therein. What is meant by “enrichment” in this context is that oAB concentrations of at least 1 g/L, preferably at least 2 g/L, are attained.

All definitions given further up in this application are also applicable to this embodiment.

In yet a further embodiment, the present application relates to a cell capable of releasing oAB from the Corynebacterium genus, preferably Corynebacterium glutamicum and more preferably Corynebacterium glutamicum ATCC13032, which differs from the wild type at least in the following features:
(i) reduced activity of anthranilate phosphoribosyltransferase compared to the respective wild type, but where residual activity must be present. The residual activity is preferably between 10% and 60%, more preferably between 20% and 50%, of the native activity in C. glutamicum ATCC13032. This is preferably achieved by reduced expression of the gene for anthranilate phosphoribosyltransferase (trpD) compared to the wild type, although expression is not completely suppressed. This is preferably effected by using a promoter sequence which has a lower transcription activity compared to the endogenous promoter sequence or by modifying the distance of the ribosome binding site to the start codon of the trpD gene or by altering the start codon itself. In a preferred embodiment of the present invention, the activity of anthranilate phosphoribosyltransferase is reduced by deletion or inactivation of the gene for endogenous anthranilate phosphoribosyltransferase (trpD) and the replacement of this gene by a gene for an anthranilate phosphoribosyltransferase having a modified ribosomal binding site and optionally a modified start codon as defined in SEQ ID NO. 1 or 2, preferably SEQ ID NO. 2. The amino acid sequence of the anthranilate phosphoribosyltransferase preferably corresponds to the endogenous anthranilate phosphoribosyltransferase, more preferably as defined by SEQ ID NO. 3 or a variant thereof.
(ii) elevated activity of shikimate kinase. This is preferably achieved by increased expression of a corresponding enzyme. In one embodiment of the present invention, the activity is increased by enhanced expression of the gene for the endogenous shikimate kinase as defined in SEQ ID NO. 6 or a variant thereof. In another preferred embodiment, this is achieved by expression of an exogenous shikimate kinase, preferably as defined in SEQ ID NO. 7 or a variant thereof. Enhanced expression of a gene can be achieved by any method known to those skilled in the art, in particular by introducing two or more copies of the corresponding gene into the microorganism or by using stronger promoters to express the endogenous enzyme. A particularly preferred promoter for expression of foreign genes or enhanced expression of endogenous genes is Ptuf as defined in SEQ ID NO. 8.
(iv) enhanced activity of 3-phosphoshikimate 1-carboxyvinyltransferase and chorismate synthase. Preferably, these enzymes have an amino acid sequence as defined in SEQ ID NO. 9 or a variant thereof and SEQ ID NO. 10 or a variant thereof. This is preferably effected by introducing additional copies of the genes coding for these enzymes into the microorganism. The Ptuf promoter is preferably used to control expression.
(iv) presence of a 3-deoxyarabinoheptulosanate-7-phosphate synthase (DAHP synthase) which is “feedback-resistant”, i.e. is not inhibited by its product or by any product formed from the product. Preference is given to an enzyme having the amino acid sequence defined in SEQ ID NO. 11 or a variant thereof.
(v) elevated activity of xylose isomerase and of xylulokinase. This elevated activity is preferably achieved by enhanced expression of both genes. This enhanced expression can be achieved by any method known to those skilled in the art, in particular by introducing two or more copies of the corresponding gene into the microorganism or by using stronger promoters to express the endogenous enzyme. Preference is given in accordance with the invention to elevated expression of enzymes having amino acid sequences as defined in SEQ ID NO. 12 or SEQ ID NO. 13/SEQ ID NO. 14, or variants thereof. Preference is given to a xylose isomerase as defined in SEQ ID NO. 12 or a variant thereof.

In a preferred embodiment of the present invention, the gene that encodes phosphoenolpyruvate carboxylase as defined in SEQ ID NO. 4 is deleted or inactivated. This may be effected in any manner familiar to those skilled in the art, preferably by deleting the gene or a partial sequence thereof, by introducing at least one stop codon or by deleting or inactivating the promoter sequence. Particular preference is given to the deletion of at least part of the protein-coding sequence of the gene (SEQ ID NO. 5).

All definitions that have been given further up in this application are also applicable to this embodiment. This is especially true of the genetic and metabolic properties of the cell.

In yet a further embodiment, the present invention relates to the use of a microbial cell that differs from the wild type in the features defined above in this application for production of oAB by a microbial fermentation with a mixture of glucose and xylose as energy source and carbon source.

All definitions that have been given further up in this application are also applicable to this embodiment. This relates more particularly to the cultivation conditions, the suitable concentrations and ratios of glucose and xylose, and the microbial cells used for microbial fermentation.

In yet another embodiment, the present invention relates to a composition comprising
a) microbial cells that are capable of releasing oAB and can convert glucose and xylose to oAB;
b) a mixture of glucose and xylose having a glucose content between 5% by weight and 86% by weight and a xylose content between 95% by weight and 14% by weight, where the proportions of glucose and xylose add up to 100%; and
c) at least one nitrogen source, at least one phosphorus source, at least one sulfur source and trace elements.

All definitions given further up in this application are also applicable to this embodiment.

The composition of the invention is a culture medium containing the microbial cells, such that compliance with the above-described incubation conditions is all that is necessary for the microbial cells to convert the glucose present and the xylose to oAB.

If the composition defined above is used as intended, the microbial cells present therein will produce oAB. Consequently, the composition, in a preferred embodiment of the present invention, additionally contains oAB. More preferably, it contains at least 1.5 g/L oAB.

The working examples which follow serve merely to illustrate the invention. They are not intended to limit the scope of protection of the claims in any way.

### EXAMPLES

All experiments except that in example 3 were conducted with the strain produced as described below.

The bacterium Corynebacterium glutamicum ATCC13032 was used as the basis for production of a microbial strain capable of utilizing xylose as carbon source for production of anthranilic acid. For this purpose, the original strain was first adapted by adaptive laboratory evolution (ALE) to rising anthranilic acid concentrations in the culture supernatant. Thereafter, this was made to produce anthranilic acid by directed chromosomal modifications. Subsequently, the strain was provided with genes from what is called the xylose isomerase pathway, since C. glutamicum ATCC13032 does not have a native metabolic pathway for utilization of xylose. All genetic modifications, i.e. chromosomal deletions and integration of genes, were effected by double homologous recombination using corresponding pk19mobsacB derivatives (Schäfer et al., 1994: “Small mobilizable multi-purpose cloning vectors derived from the Escherichia coli plasmids pk18 and pk19: selection of defined deletions in the chromosome of Corynebacterium glutamicum.” Gene 145(1):69-73. doi: 10.1016/0378-1119 (94) 90324-7).

The activity of anthranilate phosphoribosyltransferase TrpD was lowered by first deleting the native trpD allele and replacing it with an allele (called trpD5) having a GTG rather than ATG start codon and a ribosome binding site with reduced distance from the start codon (SEQ ID NO. 1).

The gene (SEQ ID NO. 5) that encodes one or the only phosphoenolpyruvate carboxylase in C. glutamicum (SEQ ID NO. 4) was deleted.

In order to boost the aromatic biosynthesis pathway, an artificial polycistronic P_{tuf}-aroLAC operon (SEQ ID NO. 15) consisting of the aroL (b0388) genes from Escherichia coli, and the aroA (cg0873) and aroC (cg1829) genes from C. glutamicum, were integrated downstream of cg2563 under the control of the constitutive promoter of the elongation factor Tuf. In addition, the aroG^{new} allele, which encodes a feedback-resistant variant of DAHP synthase (SEQ ID NO. 11) from E. coli, was integrated into the genome of the strain downstream of cg3132.

In order to render the strain capable of feeding xylose into the nonoxidative pentose phosphate pathway, a synthetic construct consisting of a codon-optimized xylose isomerase gene xylA (xcc1758) from Xanthomonas campestris pv. campestris (amino acid sequence of the enzyme according to SEQ ID NO. 13), and the xylulokinase gene xylB (cg0147) from C. glutamicum (amino acid sequence of the enzyme according to SEQ ID NO. 14) was produced, in each case under the control of the Ptuf promoter sequence (SEQ ID NO. 8) and followed by an rrnB terminator from E. coli. The construct, called P_{tuf}-xylA_{Xcc}-P_{tuf}-xylB_{Cg}-T_{mnB} (SEQ ID NO. 16), was integrated into the genome of the strain downstream of cg3344.

### Example 1

Comparative cultivations of the above-specified strain for production of ortho-aminobenzoic acid were conducted proceeding from two different sugars in each of four different ratios to one another. The main culture media were produced here such that the two sugars D-glucose and D-xylose were present in amounts as in table 1, based on the total sugar content of 20 g/L.

TABLE 1
Overview of the compositions of the two different sugars D-glucose
and D-xylose in the main culture media. For media 1-4 and hence
each ratio of amounts of sugar, four replicates were run in each
case, each with an initial cultivation volume of 50 mL.

% by wt. of sugar (based on
the total sugar content)

| Medium name | D-Glucose | D-Xylose |
|---|---|---|
| Medium 1 | 100 | 0 |
| Medium 2 | 64 | 36 |
| Medium 3 | 75 | 25 |
| Medium 4 | 88 | 12 |

For each condition and each ratio of amounts of sugar from table 1, four replicates were run. The cultivation conditions are shown below (table 2).

TABLE 2
Overview of the culture conditions for the preparatory and
main cultures. For the incubation of the second preparatory
cultures (25 mL each), Erlenmeyer flasks with a maximum capacity
of 500 mL each were used, and for the incubation of the main
cultures (initially 50 mL each before the first sampling)
Erlenmeyer flasks having a maximum capacity of 1000 mL each.
The sterile barriers used were cotton plugs.

| Parameter | Setting | Comment |
|---|---|---|
| Shake frequency (rpm) | 200 | — |
| Temperature (° C.) | 30 | — |
| Initial volume per | 25 | In 500 mL Erlenmeyer flask, |
| replicate (mL) | | for the preparatory culture |
| Initial volume per | 50 | In 1000 mL Erlenmeyer flask, |
| replicate (mL) | | for the main culture |

### Media Used, and Composition and Production Thereof

Unless stated otherwise, all media were produced with ddH₂O and autoclaved.

TABLE 3
Liquid and solid complex media composed of Brain
Heart Infusion (BHI) for growing of cells.

| Medium | Composition | Addition before/after the autoclave |
|---|---|---|
| BHI broth | 37 g/L BHI broth | Before autoclaving |
| BHI agar plates | 52 g/L BHI agar | Before autoclaving |

TABLE 4
Liquid minimal medium with complex constituents for the growing of cells in the preparatory culture. The amounts
weighed out are given for 1 L of complete CGXII preparatory culture medium. Once all the reagents have been
supplemented in the medium, the final target concentrations are attained in the complete medium.

| | | Corresponding to final | | |
|---|---|---|---|---|
| | | concentration in the | | Addition before/ |
| Medium | Composition | complete medium | | after the autoclave |
| CGXII preparatory | 1 g KH₂PO₄ | 1 | g/L | Everything hereafter |
| culture medium | 1 g K₂HPO₄ | 1 | g/L | added before autoclaving. |
| | 10 g (NH₄)₂SO₄ | 10 | g/L | |
| | 5 g urea | 5 | g/L | |
| | 62 g MOPS | 62 | g/L | |
| | 5 g yeast extract | 5 | g/L | |
| | 1 mL CaCl₂ stock solution | 1 | mL/L | |

Dissolve in 800 mL of ddH₂O, adjust pH to pH = 7.0-7.5 with KOH pellets, make up to 940 mL with ddH₂O and autoclave.

| 1.25 mL MgSO₄ stock solution | 1.25 | mL/L | Everything hereafter |
|---|---|---|---|
| | | | added after autoclaving: |
| 1 mL trace element stock solution | 1 | mL/L | |
| 1 mL biotin stock solution | 1 | mL/L | |

| | |
|---|---|
| 33 mL D-glucose stock solution OR | 20 g/L D-glucose OR |
| 33 mL D-glucose and 16.5 mL D- | 20 g/L D-glucose and |
| xylose stock solution | 10 g/L D-xylose |
| 23.75 mL water OR | — |
| 7.25 mL water | |

TABLE 5
Liquid minimal medium for the main culture. The amounts weighed out are given for 1
L of complete CGXII main culture medium. Once all the reagents have been supplemented
in the medium, the final target concentrations are attained in the complete medium.

| | | Corresponding to final | | |
|---|---|---|---|---|
| | | concentration in the | | Addition before/ |
| Medium | Composition | complete medium | | after the autoclave |
| CGXII main culture | 1 g KH₂PO₄ | 1 | g/L | Everything hereafter |
| medium | 1 g K₂HPO₄ | 1 | g/L | added before autoclaving: |
| | 10 g (NH₄)₂SO₄ | 10 | g/L | |
| | 62 g MOPS | 62 | g/L | |
| | 1 mL CaCl₂ stock solution | 1 | mL/L | |

Dissolve in 800 mL of ddH₂O, adjust pH to pH = 7.0-7.5 with KOH pellets, make up to 940 mL with ddH₂O and autoclave.

| | | | |
|---|---|---|---|
| 1.25 mL MgSO₄ stock solution | 1.25 | mL/L | Everything hereafter |
| | | | added after autoclaving: |
| 1 mL trace element stock solution | 1 | mL/L | |
| 1 mL biotin stock solution | 1 | mL/L | |

| | |
|---|---|
| 33 mL D-glucose stock solution OR | 20 g/L D-glucose OR |
| 33 mL D-xylose stock solution | 20 g/L D-xylose |
| 23.75 mL water | — |

TABLE 6
Overview of production of the 2 g/L biotin stock solution. Rather
than being autoclaved, the solution is sterile-filtered (0.2
μm). The solution can be stored at 4° C. for 1 month.

| Medium | Composition | Note |
|---|---|---|
| Biotin | 2 g/L | Solution is sterile-filtered. |
| stock | biotin | During the dissolving, the pH is measured |
| solution | | while stirring and adjusted to pH = 7.2 |
| | | with 1M KOH. |

TABLE 7
Overview of production of the 600 g/L D-glucose stock solution.

| Medium | Composition | Note |
|---|---|---|
| D-glucose | 660 g D- | Substance is dissolved in 554 g of hot |
| stock | glucose | ddH₂O. Total weight of 1 L of the complete |
| solution | monohydrate | solution: 1214 g. Boil briefly for complete |
| | | dissolution prior to autoclaving. |

TABLE 8
Overview of production of the 600 g/L D-xylose stock solution.

| Medium | Composition | Note |
|---|---|---|
| D-xylose | 600 g D- | Substance is dissolved in 595 g of hot ddH₂O. |
| stock | xylose | Total weight of 1 L of the complete solution: |
| solution | | 1195 g. Boil briefly for complete dissolution |
| | | prior to autoclaving. |

TABLE 9
Overview of production of the 10 g/L CaCl₂ stock solution.

| Medium | Composition | Note |
|---|---|---|
| CaCl₂ | 10 g/L CaCl₂ × | Solution need not be autoclaved since it is |
| stock | 2 H₂O | supplemented before the autoclaving |
| solution | | operation of the CGXII media. |

TABLE 10
Overview of production of the 200 g/L MgSO₄ stock solution.

| Medium | Composition | Note |
|---|---|---|
| MgSO₄ stock | 200 g/L MgSO₄ × 7 H₂O | Solution is sterile-filtered. |
| solution | | |

TABLE 11
Overview of production of the 1000x trace element solution. Rather
than being autoclaved, the solution is sterile-filtered (0.2 μm).
This solution has a shelf life of 6 months at 4° C. Because
of the small weights, it is advisable to produce a 1 L batch.

| Medium | Composition | Note |
|---|---|---|
| Trace | 10 g/L MnSO₄ × H₂O | Solution is sterile-filtered. |
| element | 10 g/L FeSO₄ × 7 H₂O | |
| solution | 1 g/L ZnSO₄ × 7 H₂O | |
| | 0.2 g/L CuSO₄ × 5 H₂O | |
| | 0.02 g/L NiCl₂ × 6 H₂O | |

Dissolve in ddH₂O and adjust pH with HCl to pH = 1.

TABLE 12
Overview of production of 1x phosphate buffer (PBS). 10x PBS (article
number BP399-1) from Fisher Scientific GmbH was used.

| Medium | Composition | Note |
|---|---|---|
| 1x phosphate buffer | 10x PBS | Dilute 1:10 with ddH₂O, autoclave. |

### Instruments Used

TABLE 13
Overview of the parameters examined in this study and
instruments and methods for examination thereof.

| Parameter | Instrument | Description |
|---|---|---|
| Glucose concentration | Cedex Bio Analyzer, Roche | Cedex Bio Glucose assay with |
| | | article number RD- |
| | | 06343732001, used according |
| | | to manufacturer's instructions |
| NH₃ concentration | Cedex Bio Analyzer, Roche | Cedex Bio NH3 assay with |
| | | article number RD- |
| | | 06343775001, used according |
| | | to manufacturer's instructions |
| ortho-Aminobenzoic acid | HPLC, Agilent | For separation and |
| concentration | G7104C 1260 flexible pump | quantification of ortho- |
| | G7167A 1260 multisampler | aminobenzoic acid in the |
| | G7116A 1260 multicolumn | sterile-filtered supernatant of |
| | thermostat (MCT) | the samples, an Agilent Eclipse |
| | G7117C 1260 DAD HS | Plus C18 column (4.6 × 150 mm, |
| | G7162A 1260 RID | 5 μm) and a Zorbax Eclipse Plus |
| | | C18 precolumn cartridge (4.6 × |
| | | 12 mm, 5 μm, 5 mm) were |
| | | used. The ortho-aminobenzoic |
| | | acid was detected by means of |
| | | the diode array detector (DAD). |
| D-Xylose concentration | HPLC, ROA | For separation and |
| | HPLC, Agilent | quantification of D-xylose in the |
| | G7104C 1260 flexible pump | sterile-filtered supernatant of |
| | G7167A 1260 multisampler | the samples, a Phenomenex |
| | G7116A 1260 multicolumn | Rezex ROA-Organic Acid H + 8% |
| | thermostat (MCT) | column (300 × 7.8 mm) and a |
| | G7117C 1260 DAD HS | SecurityGuard Cartridge Carbo- |
| | G7162A 1260 RID | H precolumn cartridge (4 × |
| | | 3.0 mm) were used. The D- |
| | | xylose was detected by means |
| | | of the refractive index detector |
| | | (RID). |
| Incubation of the cultures | ISF1-X shaker incubator, | |
| | Kuhner Shaker GmbH | |
| pH | SevenCompact S210 pH meter, | |
| | Mettler Toledo | |
| | InLab Expert Pro-ISM pH | |
| | electrode, Mettler Toledo | |
| Optical density (OD₆₀₀) | CO8000 cell density meter, | |
| | WPA biowave | |
| Weight of dry biomass | SARTORIUS CUBIS ® 225S Semi- | |
| | Micro Balance, Sartorius | |

### Procedure

The starter cultures were generated by taking cell mass from dormant forms in glycerol of the microbial cultures used for the culturing, and these were used to inoculate a BHI agar plate. The BHI agar plates were incubated at 30° C. for 72 h.

The agar plate culture set up in this way was used to inoculate a BHI liquid culture. For this purpose, a little cell mass was taken from the respective BHI agar plate cultures and 4 mL of liquid BHI medium in each case was inoculated in a round-bottom tube. The liquid cultures were incubated at 30° C. and 200 rpm for about 7.5 h (preparatory culture I).

For the second preparatory culture, two media with different carbon sources were used. For this purpose, CGXII preparatory culture medium was produced with either 20 g/L D-glucose or with 20 g/L D-glucose and additionally 10 g/L D-xylose. For this purpose, 23 mL in each case of the corresponding preparatory culture medium was transferred into a 500 mL Erlenmeyer flask, 2 mL of the BHI liquid preparatory culture I was added to each batch, and these two shaken flask preparatory cultures were incubated at 200 rpm and 30° C. for 17 h.

The preparatory culture with D-glucose as the sole energy source and carbon source was used for the inoculation of the main cultures with medium 1. Proceeding from the preparatory culture with 20 g/L D-glucose and additionally 10 g/L D-xylose, three different main cultures (media 2-4) were inoculated.

On completion of incubation of the second preparatory culture in the shaken flask, the optical density of the cultures was measured. Depending on the cell density achieved, a proportion of these second preparatory cultures was taken, the cells were pelletized by centrifugation and washed in sterile phosphate buffer (singly concentrated), and resuspended in 50 mL in each case of the corresponding main culture medium (media 1-4).

TABLE 14
Measurement of optical density (OD₆₀₀) on completion of incubation
of the second preparatory culture in the shaken flask. This was
used to calculate what volume of the second preparatory culture is needed
to inoculate 50 mL in each case of main culture with an initial
OD₆₀₀ of 1. For each medium, four replicates were run. Since three
xylose-containing media (media 2-4) were to be tested, a total
of 12 aliquots were taken for the inoculation from the preparatory
culture with 20 g/L D-glucose and 10 g/L D-xylose.

| | OD₆₀₀ (—) | Volume required |
|---|---|---|
| with 20 g/L D-glucose | 30 | 4 × 1.7 mL for medium 1 |
| with 20 g/L D-glucose and | 32 | 4 × 1.6 mL for medium 2 |
| 10 g/L D-xylose | | 4 × 1.6 mL for medium 3 |
| | | 4 × 1.6 mL for medium 4 |

The main cultures produced in this way were transferred to a Kuhner incubation shaker (table 13) and incubated. For the sampling over the period of cultivation, the shaken flasks were taken from the incubation shaker and weighed in order to ascertain evaporation effects. Samples were taken under sterile conditions for determination of glucose, xylose, ortho-aminobenzoic acid and dry biomass.

### Results

### D-Xylose Concentration

TABLE 15
Overview of the D-xylose concentrations over time for four
replicates in each case with medium 1 and medium 2.

| Condition | | | | | | | | |
|---|---|---|---|---|---|---|---|---|
| (% by wt. | | | | | | | | |
| of sugars | | | | | | | | |
| based on | Medium 1 | | | | Medium 2 | | | |
| total sugar) | (100% by wt. of D-glucose, | | | | (64% by wt. of D-glucose, | | | |
| Replicate | 0% by wt. of D-xylose) | | | | 36% by wt. of D-xylose) | | | |
| (—) | 1 | 2 | 3 | 4 | 1 | 2 | 3 | 4 |
| Culture | D-Xylose | D-Xylose | D-Xylose | D-Xylose | D-Xylose | D-Xylose | D-Xylose | D-Xylose |
| time (h) | (g/L) | (g/L) | (g/L) | (g/L) | (g/L) | (g/L) | (g/L) | (g/L) |
| 0.00 | 0 | 0 | 0 | 0 | 7.53 | 7.28 | 7.36 | 7.46 |
| 19.00 | 0 | 0 | 0 | 0 | 7.20 | 7.05 | 7.09 | 7.24 |
| 26.00 | 0 | 0 | 0 | 0 | 7.22 | 7.15 | 7.18 | 7.38 |
| 42.83 | 0 | 0 | 0 | 0 | 5.37 | 5.20 | 5.25 | 5.43 |
| 50.00 | 0 | 0 | 0 | 0 | 4.19 | 4.06 | 4.09 | 4.21 |
| 66.50 | 0 | 0 | 0 | 0 | 2.41 | 2.22 | 2.26 | 2.51 |

### D-Xylose Concentration

TABLE 16
Overview of the D-xylose concentrations over time for four
replicates in each case with medium 3 and medium 4.

| Condition | | | | | | | | |
|---|---|---|---|---|---|---|---|---|
| (% by wt. | | | | | | | | |
| of sugars | | | | | | | | |
| based on | Medium 3 | | | | Medium 4 | | | |
| total sugar) | (75% by wt. of D-glucose, | | | | (88% by wt. of D-glucose, | | | |
| Replicate | 25% by wt. of D-xylose) | | | | 12% by wt. of D-xylose) | | | |
| (—) | 1 | 2 | 3 | 4 | 1 | 2 | 3 | 4 |
| Culture | D-Xylose | D-Xylose | D-Xylose | D-Xylose | D-Xylose | D-Xylose | D-Xylose | D-Xylose |
| time (h) | (g/L) | (g/L) | (g/L) | (g/L) | (g/L) | (g/L) | (g/L) | (g/L) |
| 0.00 | 5.15 | 5.11 | 5.10 | 5.11 | 2.51 | 2.51 | 2.54 | 2.51 |
| 19.00 | 4.95 | 4.94 | 4.94 | 4.96 | 2.34 | 2.38 | 2.37 | 2.39 |
| 26.00 | 4.88 | 4.89 | 4.89 | 4.90 | 2.24 | 2.28 | 2.26 | 2.28 |
| 42.83 | 3.31 | 3.32 | 3.30 | 3.18 | 1.08 | 1.16 | 1.07 | 1.20 |
| 50.00 | 2.15 | 2.16 | 2.15 | 2.03 | 0.46 | 0.48 | 0.44 | 0.49 |
| 66.50 | 0.83 | 0.84 | 0.81 | 0.75 | 0.10 | 0.10 | 0.08 | 0.10 |

### D-Glucose Concentration

TABLE 17
Overview of the D-glucose concentrations over time for four
replicates in each case with medium 1 and medium 2.

| Condition | | | | | | | | |
|---|---|---|---|---|---|---|---|---|
| (% by wt. | | | | | | | | |
| of sugars | | | | | | | | |
| based on | Medium 1 | | | | Medium 2 | | | |
| total sugar) | (100% by wt. of D-glucose, | | | | (64% by wt. of D-glucose, | | | |
| Replicate | 0% by wt. of D-xylose) | | | | 36% by wt. of D-xylose) | | | |
| (—) | 1 | 2 | 3 | 4 | 1 | 2 | 3 | 4 |
| Culture | D-Glucose | D-Glucose | D-Glucose | D-Glucose | D-Glucose | D-Glucose | D-Glucose | D-Glucose |
| time (h) | (g/L) | (g/L) | (g/L) | (g/L) | (g/L) | (g/L) | (g/L) | (g/L) |
| 0.00 | 21.46 | 21.46 | 21.46 | 21.46 | 13.19 | 13.19 | 13.19 | 13.19 |
| 19.00 | 16.08 | 16.51 | 16.23 | 16.62 | 9.18 | 8.90 | 9.03 | 9.37 |
| 26.00 | 12.38 | 12.56 | 12.40 | 12.89 | 6.21 | 5.95 | 6.15 | 6.44 |
| 42.83 | 0.00 | 0.00 | 0.00 | 0.00 | 0.00 | 0.00 | 0.00 | 0.00 |
| 50.00 | 0.00 | 0.00 | 0.00 | 0.00 | 0.00 | 0.00 | 0.00 | 0.00 |
| 66.50 | 0.00 | 0.00 | 0.00 | 0.00 | 0.00 | 0.00 | 0.00 | 0.00 |

### D-Glucose Concentration

TABLE 18
Overview of the D-glucose concentrations over time for four
replicates in each case with medium 3 and medium 4.

| Condition | | | | | | | | |
|---|---|---|---|---|---|---|---|---|
| (% by wt. | | | | | | | | |
| of sugars | | | | | | | | |
| based on | Medium 3 | | | | Medium 4 | | | |
| total sugar) | (75% by wt. of D-glucose, | | | | (88% by wt. of D-glucose, | | | |
| Replicate | 25% by wt. of D-xylose) | | | | 12% by wt. of D-xylose) | | | |
| (—) | 1 | 2 | 3 | 4 | 1 | 2 | 3 | 4 |
| Culture | D-Glucose | D-Glucose | D-Glucose | D-Glucose | D-Glucose | D-Glucose | D-Glucose | D-Glucose |
| time (h) | (g/L) | (g/L) | (g/L) | (g/L) | (g/L) | (g/L) | (g/L) | (g/L) |
| 0.00 | 15.78 | 15.78 | 15.78 | 15.78 | 18.27 | 18.27 | 18.27 | 18.27 |
| 19.00 | 11.64 | 11.65 | 11.62 | 11.57 | 13.80 | 13.99 | 13.68 | 14.024 |
| 26.00 | 8.47 | 8.49 | 8.48 | 8.38 | 10.26 | 10.53 | 10.21 | 10.61937 |
| 42.83 | 0.00 | 0.00 | 0.00 | 0.00 | 0.00 | 0.00 | 0.00 | 0 |
| 50.00 | 0.00 | 0.00 | 0.00 | 0.00 | 0.00 | 0.00 | 0.00 | 0 |
| 66.50 | 0.00 | 0.00 | 0.00 | 0.00 | 0.00 | 0.00 | #NV | 0 |

### Dry Biomass Concentration (DBM)

TABLE 19
Final dry biomass concentrations in double determination for
four replicates in each case with medium 1 and medium 2.

| Condition | | | | | | | | |
|---|---|---|---|---|---|---|---|---|
| (% by wt. | | | | | | | | |
| of sugars | | | | | | | | |
| based on | Medium 1 | | | | Medium 2 | | | |
| total sugar) | (100% by wt. of D-glucose, | | | | (64% by wt. of D-glucose, | | | |
| Replicate | 0% by wt. of D-xylose) | | | | 36% by wt. of D-xylose) | | | |
| (—) | 1 | 2 | 3 | 4 | 1 | 2 | 3 | 4 |
| Culture | DBM | DBM | DBM | DBM | DBM | DBM | DBM | DBM |
| time (h) | (g/L) | (g/L) | (g/L) | (g/L) | (g/L) | (g/L) | (g/L) | (g/L) |
| 66.50 | 4.91 | 5.21 | 4.85 | 5.14 | 4.22 | 4.25 | 3.93 | 4.02 |
| 66.50 | 5.01 | 5.26 | 4.84 | 4.97 | 3.72 | 4.12 | 4.01 | 4.16 |

### Dry Biomass Concentration (DBM)

TABLE 20
Final dry biomass concentrations in double determination for
four replicates in each case with medium 3 and medium 4.

| Condition | | | | | | | | |
|---|---|---|---|---|---|---|---|---|
| (% by wt. | | | | | | | | |
| of sugars | | | | | | | | |
| based on | Medium 3 | | | | Medium 4 | | | |
| total sugar) | (75% by wt. of D-glucose, | | | | (88% by wt. of D-glucose, | | | |
| Replicate | 25% by wt. of D-xylose) | | | | 12% by wt. of D-xylose) | | | |
| (—) | 1 | 2 | 3 | 4 | 1 | 2 | 3 | 4 |
| Culture | DBM | DBM | DBM | DBM | DBM | DBM | DBM | DBM |
| time (h) | (g/L) | (g/L) | (g/L) | (g/L) | (g/L) | (g/L) | (g/L) | (g/L) |
| 66.50 | 4.61 | 4.39 | 4.46 | 4.48 | 4.76 | 4.4 | 4.7 | 4.7 |
| 66.50 | 4.49 | 4.34 | 4.41 | 4.56 | 4.67 | 4.62 | 4.81 | 4.88 |

Ortho-Aminobenzoic Acid Concentration (oAB)

TABLE 21
Overview of the ortho-aminobenzoic acid concentration over time
for four replicates in each case with medium 1 and medium 2.

| | Condition (% by wt. of sugars based on total sugar) | | | | | | | |
|---|---|---|---|---|---|---|---|---|
| | Medium 1 (100% by wt. of D-glucose, | | | | Medium 2 (64% by wt. of D-glucose, | | | |
| | 0% by wt. of D-xylose) | | | | 36% by wt. of D-xylose) | | | |
| | Replicate (—) | | | | | | | |
| | 1 | 2 | 3 | 4 | 1 | 2 | 3 | 4 |
| Culture time | oAB | oAB | oAB | oAB | oAB | oAB | oAB | oAB |
| (h) | (g/L) | (g/L) | (g/L) | (g/L) | (g/L) | (g/L) | (g/L) | (g/L) |
| 0.00 | 0.27 | 0.27 | 0.26 | 0.26 | 0.26 | 0.25 | 0.25 | 0.24 |
| 19.00 | 0.34 | 0.33 | 0.32 | 0.34 | 0.18 | 0.30 | 0.28 | 0.26 |
| 26.00 | 0.62 | 0.62 | 0.61 | 0.60 | 0.53 | 0.52 | 0.51 | 0.51 |
| 42.83 | 2.15 | 2.21 | 2.15 | 2.18 | 1.68 | 1.68 | 1.74 | 1.73 |
| 50.00 | 2.56 | 2.55 | 2.41 | 2.47 | 2.11 | 2.08 | 2.13 | 2.09 |
| 66.50 | 2.31 | 2.35 | 2.30 | 2.37 | 2.40 | 2.38 | 2.36 | 2.42 |

Ortho-Aminobenzoic Acid Concentration (oAB)

TABLE 22
Overview of the ortho-aminobenzoic acid concentration over time
for four replicates in each case with medium 3 and medium 4.

| | Condition (% by wt. of sugars based on total sugar) | | | | | | | |
|---|---|---|---|---|---|---|---|---|
| | Medium 3 (75% by wt. of D-glucose, | | | | Medium 4 (88% by wt. of D-glucose, | | | |
| | 25% by wt. of D-xylose) | | | | 12% by wt. of D-xylose) | | | |
| | Replicate (—) | | | | | | | |
| | 1 | 2 | 3 | 4 | 1 | 2 | 3 | 4 |
| Culture time | oAB | oAB | oAB | oAB | oAB | oAB | oAB | oAB |
| (h) | (g/L) | (g/L) | (g/L) | (g/L) | (g/L) | (g/L) | (g/L) | (g/L) |
| 0.00 | 0.25 | 0.26 | 0.27 | 0.27 | 0.27 | 0.27 | 0.28 | 0.26 |
| 19.00 | 0.28 | 0.27 | 0.28 | 0.28 | 0.28 | 0.28 | 0.29 | 0.27 |
| 26.00 | 0.53 | 0.53 | 0.53 | 0.54 | 0.56 | 0.56 | 0.58 | 0.55 |
| 42.83 | 1.80 | 1.81 | 1.81 | 1.94 | 2.07 | 2.02 | 2.13 | 1.97 |
| 50.00 | 2.39 | 2.36 | 2.39 | 2.41 | 2.41 | 2.43 | 2.48 | 2.52 |
| 66.50 | 2.51 | 2.50 | 2.51 | 2.62 | 2.46 | 2.48 | 2.46 | 2.43 |

### Final Product Yield

TABLE 23
Overview of the ortho-aminobenzoic acid yields after 66.50 h for four replicates with medium
1 and medium 2. These are reported as the mass-based (g_{oAB}/g_{C source}) and molar amount-based quotient
(mol_{oAB}/mol_{C source}), including firstly the metabolized carbon source and secondly the carbon
source included in the initial charge of medium in the calculation.

| | Condition (% by wt. of sugars based on total sugar) | | | | | | | |
|---|---|---|---|---|---|---|---|---|
| | Medium 1 (100% by wt. of D-glucose, | | | | Medium 2 (64% by wt. of D-glucose, | | | |
| | 0% by wt. of D-xylose) | | | | 36% by wt. of D-xylose) | | | |
| | Replicate (—) | | | | | | | |
| | 1 | 2 | 3 | 4 | 1 | 2 | 3 | 4 |
| Yield after | 0.0857 | 0.0881 | 0.0860 | 0.0900 | 0.1054 | 0.1044 | 0.1035 | 0.1090 |
| cultivation for | | | | | | | | |
| 66.50 h (g_{oAB}/ | | | | | | | | |
| g_{C source consumed}) | | | | | | | | |
| Yield after | 0.0819 | 0.0841 | 0.0822 | 0.0858 | 0.0890 | 0.0890 | 0.0880 | 0.0914 |
| cultivation for | | | | | | | | |
| 66.50 h (g_{oAB}/ | | | | | | | | |
| g_{C source initially charged}) | | | | | | | | |
| Yield after | 0.1126 | 0.1157 | 0.1130 | 0.1132 | 0.1312 | 0.1300 | 0.1288 | 0.1359 |
| cultivation for | | | | | | | | |
| 66.50 h (mol_{oAB}/ | | | | | | | | |
| mol_{C source consumed}) | | | | | | | | |
| Yield after | 0.1075 | 0.1105 | 0.1079 | 0.1128 | 0.1090 | 0.1091 | 0.1079 | 0.1120 |
| cultivation for | | | | | | | | |
| 66.50 h (mol_{oAB}/ | | | | | | | | |
| mol_{C source initially charged}) | | | | | | | | |

### Final Product Yield

TABLE 24
Overview of the ortho-aminobenzoic acid yields after 66.50 h for four replicates with medium
3 and medium 4. These are reported as the mass-based (g_{oAB}/g_{C source}) and molar amount-based quotient
(mol_{oAB}/mol_{C source}), including firstly the metabolized carbon source and secondly the carbon
source included in the initial charge of medium in the calculation.

| | Condition (% by wt. of sugars based on total sugar) | | | | | | | |
|---|---|---|---|---|---|---|---|---|
| | Medium 3 (75% by wt. of D-glucose, | | | | Medium 4 (88% by wt. of D-glucose, | | | |
| | 25% by wt. of D-xylose) | | | | 12% by wt. of D-xylose) | | | |
| | Replicate (—) | | | | | | | |
| | 1 | 2 | 3 | 4 | 1 | 2 | 3 | 4 |
| Yield after | 0.1020 | 0.0999 | 0.1017 | 0.1055 | 0.0949 | 0.0967 | 0.0943 | 0.0949 |
| cultivation for | | | | | | | | |
| 66.50 h (g_{oAB}/ | | | | | | | | |
| g_{C source consumed}) | | | | | | | | |
| Yield after | 0.0933 | 0.0927 | 0.0930 | 0.0968 | 0.0901 | 0.0917 | 0.0865 | 0.0900 |
| cultivation for | | | | | | | | |
| 66.50 h (g_{oAB}/ | | | | | | | | |
| g_{C source initially charged}) | | | | | | | | |
| Yield after | 0.1286 | 0.1253 | 0.1282 | 0.1329 | 0.1219 | 0.1242 | 0.1210 | 0.1219 |
| cultivation for | | | | | | | | |
| 66.50 h (mol_{oAB}/ | | | | | | | | |
| mol_{C source consumed}) | | | | | | | | |
| Yield after | 0.1168 | 0.1161 | 0.1165 | 0.1212 | 0.1155 | 0.1176 | 0.1147 | 0.1154 |
| cultivation for | | | | | | | | |
| 66.50 h (mol_{oAB}/ | | | | | | | | |
| mol_{C source initially charged}) | | | | | | | | |

### Averages of the Final Product Yields and Standard Deviations

TABLE 25
Overview of the averages and standard deviations of the ortho-aminobenzoic
acid yields from four replicates in each case after 66.50 h. These are
reported as the mass-based (g_{oAB}/g_{C source}) and molar amount-based quotient
(mol_{oAB}/mol_{C source}), including firstly the metabolized carbon source and
secondly the carbon source included in the initial charge of medium in the calculation.

| Medium No. (—) | Medium 1 | Medium 2 | Medium 3 | Medium 4 |
|---|---|---|---|---|
| Condition (% by wt. | 100% by wt. of | 64% by wt. of | 75% by wt. of | 88% by wt. of |
| of sugars based on | D-glucose, 0% | D-glucose, 36% | D-glucose, 25% | D-glucose, 12% |
| total sugar) | by wt. of D- | by wt. of D- | by wt. of D- | by wt. of D- |
| | xylose | xylose | xylose | xylose |
| Average yield after | 0.087 ± 0.002 | 0.106 ± 0.002 | 0.102 ± 0.002 | 0.095 ± 0.001 |
| cultivation for 66.50 h | | | | |
| (g_{oAB}/g_{C source consumed}) | | | | |
| Average yield after | 0.083 ± 0.002 | 0.089 ± 0.001 | 0.094 ± 0.002 | 0.090 ± 0.001 |
| cultivation for 66.50 h | | | | |
| (g_{oAB}/g_{C source initially charged}) | | | | |
| Average yield after | 0.114 ± 0.001 | 0.131 ± 0.003 | 0.129 ± 0.003 | 0.122 ± 0.001 |
| cultivation for 66.50 h | | | | |
| (mol_{oAB}/mol_{C source consumed}) | | | | |
| Average yield after | 0.110 ± 0.002 | 0.110 ± 0.002 | 0.118 ± 0.002 | 0.116 ± 0.001 |
| cultivation for 66.50 h | | | | |
| (mol_{oAB}/mol_{C source initially charged}) | | | | |

### SUMMARY

In the shaken flask culturing described here of a xylose-metabolizing ortho-aminobenzoic acid producer based on C. glutamicum, the effect of various mixing ratios of glucose and xylose on the yield of ortho-aminobenzoic acid was examined.

By the time the cultures had ended, the xylose was in most cases still not yet fully consumed, and therefore a distinction was made in respect of the final product yield between the “substrate yield” based on the mass or molar amount of substrate consumed (g_{oAB}/g_{C source consumed} or mol_{oAB}/mol_{C source consumed}) and the “process yield” based on the mass or molar amount of the substrate initially charged (g_{oAB}/g_{C source initially charged} or mol_{oAB}/mol_{C source initially charged}).

It was found that the substrate yield was at its highest with use of medium 2 at 0.106 g_{oAB}/g_{C source consumed} or 0.129 mol_{oAB}/mol_{C source consumed}. The dry biomass concentration attained decreased with increasing xylose content. In respect of process yield, a maximum of 0.094 g_{oAB}/g_{C source initially charged} or 0.118 mol_{oAB}/mol_{C source initially charged} was achieved with medium 3, in which the energy source and carbon source used was composed to an extent of 76% by weight of glucose and 24% by weight of xylose.

### Example 2

Comparative cultivations of the above-described strain for production of ortho-aminobenzoic acid were conducted proceeding from two different sugars in eight different ratios. The main culture media were produced here such that the two sugars D-glucose and D-xylose were present in amounts as in table 26, based on the total sugar content of 20 g/L.

TABLE 26
Overview of the compositions of the different
sugars in the main cultivation media used.

% by wt. of sugar (based
on the total sugar content)

| Medium name | D-Glucose | D-Xylose |
|---|---|---|
| Medium 1 | 100 | 0 |
| Medium 2 | 86 | 14 |
| Medium 3 | 76 | 24 |
| Medium 4 | 63 | 37 |
| Medium 5 | 50 | 50 |
| Medium 6 | 39 | 61 |
| Medium 7 | 27 | 73 |
| Medium 8 | 14 | 86 |

For each condition and each ratio of amounts of sugar, four replicates were run. The cultivation conditions are shown below.

TABLE 27
Settings on the BioLector Pro for the cultivation
of the 48-well microtiter plate.

| Parameter | Setting |
|---|---|
| Temperature (° C.) | 30 |
| Shake frequency (rpm) | 1000 |
| Culture time (h) | 70.79 |
| Air humidity (%) | 85 |
| Time between measurement cycles (min) | 5 |
| Oxygen in cultivation chamber (% by wt.) | 21 |

TABLE 28
Layout of the culture plate of the M2P-MTP-48-BOH2 type. The same
volume of 1 mL of the main culture was used at each position.

| | 1 | 2 | 3 | 4 | 5 | 6 | 7 | 8 |
|---|---|---|---|---|---|---|---|---|
| A | Medium 1 | Medium 1 | Medium 1 | Medium 1 | Medium 1 | Medium 1 | Medium 2 | Medium 2 |
| B | Medium 2 | Medium 2 | Medium 2 | Medium 2 | Medium 3 | Medium 3 | Medium 3 | Medium 3 |
| C | Medium 3 | Medium 3 | Medium 4 | Medium 4 | Medium 4 | Medium 4 | Medium 4 | Medium 4 |
| D | Medium 5 | Medium 5 | Medium 5 | Medium 5 | Medium 5 | Medium 5 | Medium 6 | Medium 6 |
| E | Medium 6 | Medium 6 | Medium 6 | Medium 6 | Medium 7 | Medium 7 | Medium 7 | Medium 7 |
| F | Medium 7 | Medium 7 | Medium 8 | Medium 8 | Medium 8 | Medium 8 | Medium 8 | Medium 8 |

### Media Used, and Composition and Production Thereof

Unless stated otherwise, all media were produced with ddH₂O and autoclaved.

TABLE 29
Liquid and solid complex media composed of Brain
Heart Infusion (BHI) for growing of cells.

| | | Addition before/ |
|---|---|---|
| Medium | Composition | after the autoclave |
| BHI broth | 37 g/L BHI broth | Before autoclaving |
| BHI agar plates | 52 g/L BHI agar | Before autoclaving |

TABLE 30
Liquid minimal medium with complex constituents for the growing of cells in the
preparatory culture. The amounts weighed out are given for 1 L of complete CGXII
preparatory culture medium. Once all the reagents have been supplemented in the
medium, the final target concentrations are attained in the complete medium.

| | | Corresponding to final | | |
|---|---|---|---|---|
| | | concentration in the | | Addition before/ |
| Medium | Composition | complete medium | | after the autoclave |
| CGXII preparatory | 1 g KH₂PO₄ | 1 | g/L | Everything hereafter |
| culture medium | 1 g K₂HPO₄ | 1 | g/L | added before autoclaving. |
| | 10 g (NH₄)₂SO₄ | 10 | g/L | |
| | 5 g urea | 5 | g/L | |
| | 62 g MOPS | 62 | g/L | |
| | 5 g yeast extract | 5 | g/L | |
| | 1 mL CaCl₂ stock solution | 1 | mL/L | |

Dissolve in 800 mL of ddH₂O, adjust pH to pH = 7.0-7.5 with KOH pellets, make up to 940 mL with ddH₂O
and autoclave.

| | | | |
|---|---|---|---|
| 1.25 mL MgSO₄ stock solution | 1.25 | ml/L | Everything hereafter |
| | | | added after autoclaving: |
| 1 mL trace element stock solution | 1 | mL/L | |
| 1 mL biotin stock solution | 1 | mL/L | |

| | |
|---|---|
| 33 mL D-glucose stock solution OR | 20 g/L D-glucose OR |
| 33 mL D-glucose and | 20 g/L D-glucose and |
| 16.5 mL D-xylose stock solution | 10 g/L D-xylose |
| 23.75 mL water OR | — |
| 7.25 mL water | |

TABLE 31
Liquid minimal medium for the main culture. The amounts weighed out are given for 1
L of complete CGXII main culture medium. Once all the reagents have been supplemented
in the medium, the final target concentrations are attained in the complete medium.

| | | Corresponding to final | | |
|---|---|---|---|---|
| | | concentration in the | | Addition before/ |
| Medium | Composition | complete medium | | after the autoclave |
| CGXII main culture | 1 g KH₂PO₄ | 1 | g/L | Everything hereafter |
| medium | 1 g K₂HPO₄ | 1 | g/L | added before autoclaving: |
| | 10 g (NH₄)₂SO₄ | 10 | g/L | |
| | 62 g MOPS | 62 | g/L | |
| | 1 mL CaCl₂ stock solution | 1 | mL/L | |

Dissolve in 800 ml of ddH₂O, adjust pH to pH = 7.0-7.5 with KOH pellets, make up to 940 mL
with ddH₂O and autoclave.

| | | | |
|---|---|---|---|
| 1.25 mL MgSO₄ stock solution | 1.25 | mL/L | Everything hereafter |
| 1 mL trace element stock solution | 1 | mL/L | added after autoclaving: |
| 1 mL biotin stock solution | 1 | mL/L | |

| | |
|---|---|
| 33 mL D-glucose stock solution OR | 20 g/L D-glucose OR |
| 33 mL D-xylose stock solution | 20 g/L D-xylose |
| 23.75 mL water | — |

TABLE 32
Overview of production of the 2 g/L biotin stock solution. Rather
than being autoclaved, the solution is sterile-filtered (0.2
μm). The solution can be stored at 4° C. for 1 month.

| Medium | Composition | Note |
|---|---|---|
| Biotin stock solution | 2 g/L biotin | Solution is sterile-filtered. |
| | | During the dissolving, the pH is |
| | | measured while stirring and |
| | | adjusted to pH = 7.2 with 1M KOH. |

TABLE 33
Overview of production of the 600 g/L D-glucose stock solution.

| Medium | Composition | Note |
|---|---|---|
| D-glucose stock | 660 g D-glucose | Substance is dissolved in 554 g of hot ddH₂O. Total |
| solution | monohydrate | weight of 1 L of the complete solution: 1214 g. Boil |
| | | briefly for complete dissolution prior to autoclaving. |

TABLE 34
Overview of production of the 600 g/L D-xylose stock solution.

| Medium | Composition | Note |
|---|---|---|
| D-xylose | 600 g | Substance is dissolved in 595 g of hot ddH₂O. |
| stock | D-xylose | Total weight of 1 L of the complete solution: |
| solution | | 1195 g. Boil briefly for complete dissolution |
| | | prior to autoclaving. |

TABLE 35
Overview of production of the 10 g/L CaCl₂ stock solution.

| Medium | Composition | Note |
|---|---|---|
| CaCl₂ stock | 10 g/L CaCl₂ × | Solution need not be autoclaved since it |
| solution | 2 H₂O | is supplemented before the autoclaving |
| | | operation of the CGXII media. |

TABLE 36
Overview of production of the 200 g/L MgSO₄ stock solution.

| Medium | Composition | Note |
|---|---|---|
| MgSO₄ stock | 200 g/L MgSO₄ × 7 H₂O | Solution is sterile-filtered. |
| solution | | |

TABLE 37
Overview of production of the 1000x trace element solution. Rather
than being autoclaved, the solution is sterile-filtered (0.2 μm).
This solution has a shelf life of 6 months at 4° C. Because
of the small weights, it is advisable to produce a 1 L batch.

| Medium | Composition | Note |
|---|---|---|
| Trace element | 10 g/L MnSO₄ × H₂O | Solution is sterile-filtered. |
| solution | 10 g/L FeSO₄ × 7 H₂O | |
| | 1 g/L ZnSO₄ × 7 H₂O | |
| | 0.2 g/L CuSO₄ × 5 H₂O | |
| | 0.02 g/L NiCl₂ × 6 H₂O | |

Dissolve in ddH₂O and adjust pH with HCl to pH = 1.

TABLE 38
Overview of production of 1x phosphate buffer (PBS). 10x PBS (article
number BP399-1) from Fisher Scientific GmbH was used.

| Medium | Composition | Note |
|---|---|---|
| 1x phosphate buffer | 10x PBS | Dilute 1:10 with ddH₂O, autoclave. |

### Instruments Used

TABLE 39
Overview of the parameters examined in this study and
instruments and methods for examination thereof.

| Parameter | Instrument | Description |
|---|---|---|
| Glucose concentration | Cedex Bio Analyzer, Roche | Cedex Bio Glucose assay with |
| | | article number RD- |
| | | 06343732001, used according |
| | | to manufacturer's instructions |
| NH₃ concentration | Cedex Bio Analyzer, Roche | Cedex Bio NH3 assay with |
| | | article number RD- |
| | | 06343775001, used according |
| | | to manufacturer's instructions |
| ortho-Aminobenzoic acid | HPLC, Agilent | For separation and |
| concentration | G7104C 1260 flexible pump | quantification of ortho- |
| | G7167A 1260 multisampler | aminobenzoic acid in the |
| | G7116A 1260 multicolumn | sterile-filtered supernatant of |
| | thermostat (MCT) | the samples, an Agilent Eclipse |
| | G7117C 1260 DAD HS | Plus C18 column (4.6 × |
| | G7162A 1260 RID | 150 mm, 5 μm) and a Zorbax |
| | | Eclipse Plus C18 precolumn |
| | | cartridge (4.6 × 12 mm, 5 μm, |
| | | 5 mm) were used. The ortho- |
| | | aminobenzoic acid was |
| | | detected by means of the |
| | | diode array detector (DAD). |
| D-Xylose concentration | HPLC, ROA | For separation and |
| | HPLC, Agilent | quantification of D-xylose in the |
| | G7104C 1260 flexible pump | sterile-filtered supernatant of |
| | G7167A 1260 multisampler | the samples, a Phenomenex |
| | G7116A 1260 multicolumn | Rezex ROA-Organic Acid H+ 8 |
| | thermostat (MCT) | column (300 × 7.8 mm, S/N |
| | G7117C 1260 DAD HS | H20-094694) and a |
| | G7162A 1260 RID | SecurityGuard Cartridge Carbo- |
| | | H precolumn cartridge (4 × |
| | | 3.0 mm) were used. The D- |
| | | xylose was detected by means |
| | | of the refractive index detector |
| | | (RID). |
| pH | SevenCompact S210 pH | |
| | meter, Mettler Toledo | |
| | InLab Expert Pro-ISM pH | |
| | electrode, Mettler Toledo | |
| Weight of dry biomass | SARTORIUS CUBIS ® 225S | |
| | Semi-Micro Balance, | |
| | Sartorius | |

TABLE 40
Overview of the products from Beckman Coulter GmbH
used for the culturing of the main cultures.

| Product | Description | Properties |
|---|---|---|
| G-BLMF100 | BioLector ® Pro system | Integrated microfluidic system |
| | | for active pH control and |
| | | substrate feeding in the |
| | | microtiter plate, 6 filters for the |
| | | following parameters: biomass, |
| | | pH, DO (dissolved oxygen), |
| | | riboflavin (Ex 436 nm/Em 540 |
| | | nm), LG1 (pH) and RF (DO); |
| | | incubation chamber is |
| | | equipped with sensors for |
| | | moisture and temperature; |
| | | moistening function for the |
| | | incubation chamber (>75% by |
| | | weight) |
| E-O2-100 | O2 enrichment module | Module for enrichment of |
| | Rev10.3 | oxygen (up to 35% by weight), |
| | | including O2 sensor and |
| | | pressure reducer |
| E-OP-498 | Configurable LED module BL II and Pro | Configurable filter module for |
| | | BioLector ® II and Pro: The |
| | | wavelengths for emission and |
| | | excitation may be between 365 |
| | | and 800 nm (bandpass filter: |
| | | 10 nm). |
| M2P-MTP-48-BOH2 | FlowerPlate MTP, pH/DO type 2 | 48-well FlowerPlate microtiter |
| | (LG1/RF) | plate, transparent base, pH and |
| | | DO optode type 2 (LG1/RF). |
| M2P-F-GPR48-10 | Sealing foil, Gas-permeable, Reduced | Self-adhesive, gas-permeable |
| | evaporation | sealing film, serves as sterile |
| | | barrier and reduces evaporation |

### Procedure

A starter culture was generated by taking cell mass from dormant forms in glycerol of the microbial cultures used for the culturing, and these were used to inoculate a BHI agar plate. The BHI agar plate was incubated at 30° C. for 48 h.

The agar plate culture set up in this way was used to inoculate a BHI liquid culture. For this purpose, a little cell mass was taken from the BHI agar plate culture and 4 mL of liquid BHI medium was inoculated in a round-bottom tube. The liquid culture was incubated at 30° C. and 200 rpm for about 7.5 h (preparatory culture I).

For the second preparatory culture, two media with different carbon sources were used. For this purpose, CGXII preparatory culture medium was produced with either 20 g/L D-glucose or with 20 g/L D-glucose and additionally 10 g/L D-xylose, and 50 mL in each case was transferred into a 1 L Erlenmeyer flask. 2 mL of the liquid BHI preparatory culture was added to each of the two batches, and these two shaken flask preparatory cultures were incubated at 200 rpm and 30° C. for 17 h.

On completion of incubation of the second preparatory culture in the shaken flask, the optical density of the cultures, which either had only glucose available or had a mixture of glucose and xylose available as the carbon source, was measured. Depending on the cell density achieved, a proportion of these second preparatory cultures was taken, the cells were pelletized by centrifugation and washed in sterile phosphate buffer (singly concentrated), and resuspended in appropriate main culture medium (media 1-8).

TABLE 41
Measurement of optical density (OD₆₀₀) on completion of incubation
of the second preparatory culture in the shaken flask. The optical
density measured was used to calculate what volume of the second
preparatory culture is needed to inoculate 10 mL of main culture
with an initial OD₆₀₀ of 1. Since xylose-containing media with
seven different xylose concentrations were to be tested, seven
aliquots were taken for the inoculation from the preparatory
culture with 20 g/L D-glucose and 10 g/L D-xylose.

| | OD₆₀₀ (—) | Volume required |
|---|---|---|
| 20 g/L D-glucose | 23.24 | 1 × 430 μL for medium 1 |
| 20 g/L D-glucose and | 18.17 | 7 × 550 for media 2-8 |
| 10 g/L D-xylose | | |

The preparatory culture with glucose as the sole carbon source and energy source was used for the production of the main culture with medium 1. By means of the preparatory culture with glucose and xylose, the main cultures were inoculated with media 2-8. In each case 6×1 mL (6 replicates for each ratio of amounts of sugar) of the main cultures produced in this way were pipetted into different positions of a culture plate, and the culture plate was sealed with a film and transferred into the BioLector Pro.

### Results

### D-Xylose Concentration

TABLE 42
Overview of the D-xylose concentrations for the six replicates with
medium 1 and medium 2 at the start of cultivation and after 70.79 h.

| | Condition (% by wt. of sugars based on total sugar) | | | | | | | | | | | |
|---|---|---|---|---|---|---|---|---|---|---|---|---|
| | Medium 1 (100% by wt. of D-glucose, | | | | | | Medium 2 (86% by wt. of D-glucose, | | | | | |
| | 0% by wt. of D-xylose) | | | | | | 14% by wt. of D-xylose) | | | | | |
| | Position in MTP | | | | | | | | | | | |
| | A1 | A2 | A3 | A4 | A5 | A6 | A7 | A8 | B1 | B2 | B3 | B4 |
| D-Xylose after | 0 | 0 | 0 | 0 | 0 | 0 | 2.983 | 2.983 | 2.983 | 2.983 | 2.983 | 2.983 |
| cultivation for | | | | | | | | | | | | |
| 0.00 h (g/L) | | | | | | | | | | | | |
| D-Xylose after | 0 | 0 | 0 | 0 | 0 | 0 | 0.041 | 0.044 | 0.051 | 0.049 | 0.044 | 0.200 |
| cultivation for | | | | | | | | | | | | |
| 70.79 h (g/L) | | | | | | | | | | | | |

### D-Xylose Concentration

TABLE 43
Overview of the D-xylose concentrations for the six replicates with
medium 3 and medium 4 at the start of cultivation and after 70.79 h.

| | Condition (% by wt. of sugars based on total sugar) | | | | | | | | | | | |
|---|---|---|---|---|---|---|---|---|---|---|---|---|
| | Medium 3 (76% by wt. of D-glucose, | | | | | | Medium 4 (63% by wt. of D-glucose, | | | | | |
| | 24% by wt. of D-xylose) | | | | | | 37% by wt. of D-xylose) | | | | | |
| | Position in MTP | | | | | | | | | | | |
| | B5 | B6 | B7 | B8 | C1 | C2 | C3 | C4 | C5 | C6 | C7 | C8 |
| D-Xylose after | 4.884 | 4.884 | 4.884 | 4.884 | 4.884 | 4.884 | 7.567 | 7.567 | 7.567 | 7.567 | 7.567 | 7.567 |
| cultivation for | | | | | | | | | | | | |
| 0.00 h (g/L) | | | | | | | | | | | | |
| D-Xylose after | 0.398 | 0.567 | 0.549 | 0.522 | 0.410 | 0.740 | 1.809 | 1.694 | 2.164 | 2.160 | 2.086 | 2.440 |
| cultivation for | | | | | | | | | | | | |
| 70.79 h (g/L) | | | | | | | | | | | | |

### D-Xylose Concentration

TABLE 44
Overview of the D-xylose concentrations for the six replicates with
medium 1 and medium 2 at the start of cultivation and after 70.79 h.

| | Condition (% by wt. of sugars based on total sugar) | | | | | | | | | | | |
|---|---|---|---|---|---|---|---|---|---|---|---|---|
| | Medium 5 (50% by wt. of D-glucose, | | | | | | Medium 6 (39% by wt. of D-glucose, | | | | | |
| | 50% by wt. of D-xylose) | | | | | | 61% by wt. of D-xylose) | | | | | |
| | Position in MTP | | | | | | | | | | | |
| | D1 | D2 | D3 | D4 | D5 | D6 | D7 | D8 | E1 | E2 | E3 | E4 |
| D-Xylose after | 10.39 | 10.39 | 10.39 | 10.39 | 10.39 | 10.39 | 12.29 | 12.29 | 12.29 | 12.29 | 12.29 | 12.29 |
| cultivation for | | | | | | | | | | | | |
| 0.00 h (g/L) | | | | | | | | | | | | |
| D-Xylose after | 4.46 | 4.53 | 4.45 | 4.30 | 4.56 | 4.68 | 7.23 | 7.34 | 7.3 | 7.41 | 7.52 | 7.85 |
| cultivation for | | | | | | | | | | | | |
| 70.79 h (g/L) | | | | | | | | | | | | |

### D-Xylose Concentration

TABLE 45
Overview of the D-xylose concentrations for the six replicates with
medium 7 and medium 8 at the start of cultivation and after 70.79 h.

| | Condition (% by wt. of sugars based on total sugar) | | | | | | | | | | | |
|---|---|---|---|---|---|---|---|---|---|---|---|---|
| | Medium 7 (27% by wt. of D-glucose, | | | | | | Medium 8 (14% by wt. of D-glucose, | | | | | |
| | 73% by wt. of D-xylose) | | | | | | 86% by wt. of D-xylose) | | | | | |
| | Position in MTP | | | | | | | | | | | |
| | E5 | E6 | E7 | E8 | F1 | F2 | F3 | F4 | F5 | F6 | F7 | F8 |
| D-Xylose after | 14.42 | 14.42 | 14.42 | 14.42 | 14.42 | 14.42 | 16.76 | 16.76 | 16.76 | 16.76 | 16.76 | 16.76 |
| cultivation for | | | | | | | | | | | | |
| 0.00 h (g/L) | | | | | | | | | | | | |
| D-Xylose after | 10.79 | 10.66 | 10.68 | 10.77 | 10.73 | 10.19 | 14.33 | 14.73 | 14.51 | 14.65 | 14.68 | 15.47 |
| cultivation for | | | | | | | | | | | | |
| 70.79 h (g/L) | | | | | | | | | | | | |

### D-Glucose Concentration

TABLE 46
Overview of the D-glucose concentrations for the six replicates with
medium 1 and medium 2 at the start of cultivation and after 70.79 h.

| | Condition (% by wt. of sugars based on total sugar) | | | | | | | | | | | |
|---|---|---|---|---|---|---|---|---|---|---|---|---|
| | Medium 1 (100% by wt. of D-glucose, | | | | | | Medium 2 (86% by wt. of D-glucose, | | | | | |
| | 0% by wt. of D-xylose) | | | | | | 14% by wt. of D-xylose) | | | | | |
| | Position in MTP | | | | | | | | | | | |
| | A1 | A2 | A3 | A4 | A5 | A6 | A7 | A8 | B1 | B2 | B3 | B4 |
| D-Glucose | 21.05 | 21.05 | 21.05 | 21.05 | 21.05 | 21.05 | 18.31 | 18.31 | 18.31 | 18.31 | 18.31 | 18.31 |
| after | | | | | | | | | | | | |
| cultivation for | | | | | | | | | | | | |
| 0.00 h (g/L) | | | | | | | | | | | | |
| D-Glucose | 0 | 0 | 0 | 0 | 0 | 0 | 0 | 0 | 0 | 0 | 0 | 0 |
| after | | | | | | | | | | | | |
| cultivation for | | | | | | | | | | | | |
| 70.79 h (g/L) | | | | | | | | | | | | |

### D-Glucose Concentration

TABLE 47
Overview of the D-glucose concentrations for the six replicates with
medium 3 and medium 4 at the start of cultivation and after 70.79 h.

| | Condition (% by wt. of sugars based on total sugar) | | | | | | | | | | | |
|---|---|---|---|---|---|---|---|---|---|---|---|---|
| | Medium 3 (76% by wt. of D-glucose, | | | | | | Medium 4 (63% by wt. of D-glucose, | | | | | |
| | 24% by wt. of D-xylose) | | | | | | 37% by wt. of D-xylose) | | | | | |
| | Position in MTP | | | | | | | | | | | |
| | B5 | B6 | B7 | B8 | C1 | C2 | C3 | C4 | C5 | C6 | C7 | C8 |
| D-Glucose | 15.74 | 15.74 | 15.74 | 15.74 | 15.74 | 15.74 | 13.06 | 13.06 | 13.06 | 13.06 | 13.06 | 13.06 |
| after | | | | | | | | | | | | |
| cultivation for | | | | | | | | | | | | |
| 0.00 h (g/L) | | | | | | | | | | | | |
| D-Glucose | 0 | 0 | 0 | 0 | 0 | 0 | 0 | 0 | 0 | 0 | 0 | 0 |
| after | | | | | | | | | | | | |
| cultivation for | | | | | | | | | | | | |
| 70.79 h (g/L) | | | | | | | | | | | | |

### D-Glucose Concentration

TABLE 48
Overview of the D-glucose concentrations for the six replicates with
medium 5 and medium 6 at the start of cultivation and after 70.79 h.

| | Condition (% by wt. of sugars based on total sugar) | | | | | | | | | | | |
|---|---|---|---|---|---|---|---|---|---|---|---|---|
| | Medium 5 (50% by wt. of D-glucose, | | | | | | Medium 6 (39% by wt. of D-glucose, | | | | | |
| | 50% by wt. of D-xylose) | | | | | | 61% by wt. of D-xylose) | | | | | |
| | Position in MTP | | | | | | | | | | | |
| | D1 | D2 | D3 | D4 | D5 | D6 | D7 | D8 | E1 | E2 | E3 | E4 |
| D-Glucose after | 10.46 | 10.46 | 10.46 | 10.46 | 10.46 | 10.46 | 7.81 | 7.81 | 7.81 | 7.81 | 7.81 | 7.81 |
| cultivation for | | | | | | | | | | | | |
| 0.00 h (g/L) | | | | | | | | | | | | |
| D-Glucose after | 0 | 0 | 0 | 0 | 0 | 0 | 0 | 0 | 0 | 0 | 0 | 0 |
| cultivation for | | | | | | | | | | | | |
| 70.79 h (g/L) | | | | | | | | | | | | |

### D-Glucose Concentration

TABLE 49
Overview of the D-glucose concentrations for the six replicates with
medium 7 and medium 8 at the start of cultivation and after 70.79 h.

| | Condition (% by wt. of sugars based on total sugar) | | | | | | | | | | | |
|---|---|---|---|---|---|---|---|---|---|---|---|---|
| | Medium 7 (27% by wt. of D-glucose, | | | | | | Medium 8 (14% by wt. of D-glucose, | | | | | |
| | 73% by wt. of D-xylose) | | | | | | 86% by wt. of D-xylose) | | | | | |
| | Position in MTP | | | | | | | | | | | |
| | E5 | E6 | E7 | E8 | F1 | F2 | F3 | F4 | F5 | F6 | F7 | F8 |
| D-Glucose after | 5.25 | 5.25 | 5.25 | 5.25 | 5.25 | 5.25 | 2.63 | 2.63 | 2.63 | 2.63 | 2.63 | 2.63 |
| cultivation for | | | | | | | | | | | | |
| 0.00 h (g/L) | | | | | | | | | | | | |
| D-Glucose after | 0 | 0 | 0 | 0 | 0 | 0 | 0 | 0 | 0 | 0 | 0 | 0 |
| cultivation for | | | | | | | | | | | | |
| 70.79 h (g/L) | | | | | | | | | | | | |

### Dry Biomass Concentration (DBM)

TABLE 50
Overview of the dry biomass concentrations for the six
replicates with medium 1 and medium 2 after 70.79 h.

| | Condition (% by wt. of sugars based on total sugar) | | | | | | | | | | | |
|---|---|---|---|---|---|---|---|---|---|---|---|---|
| | Medium 1 (100% by wt. of D-glucose, | | | | | | Medium 2 (86% by wt. of D-glucose, | | | | | |
| | 0% by wt. of D-xylose) | | | | | | 14% by wt. of D-xylose) | | | | | |
| | Position in MTP | | | | | | | | | | | |
| | A1 | A2 | A3 | A4 | A5 | A6 | A7 | A8 | B1 | B2 | B3 | B4 |
| DBM after | 6.71 | 5.72 | 5.63 | 5.69 | 6.12 | 5.96 | 5.05 | 5.09 | 4.56 | 5.20 | 5.49 | 4.76 |
| cultivation for | | | | | | | | | | | | |
| 70.79 h (g/L) | | | | | | | | | | | | |

### Dry Biomass Concentration (DBM)

TABLE 51
Overview of the dry biomass concentrations for the six
replicates with medium 3 and medium 4 after 70.79 h.

| | Condition (% by wt. of sugars based on total sugar) | | | | | | | | | | | |
|---|---|---|---|---|---|---|---|---|---|---|---|---|
| | Medium 3 (76% by wt. of D-glucose, | | | | | | Medium 4 (63% by wt. of D-glucose, | | | | | |
| | 24% by wt. of D-xylose) | | | | | | 37% by wt. of D-xylose) | | | | | |
| | Position in MTP | | | | | | | | | | | |
| | B5 | B6 | B7 | B8 | C1 | C2 | C3 | C4 | C5 | C6 | C7 | C8 |
| DBM after | 5.37 | 4.95 | 5.36 | 4.55 | 5.37 | 6.17 | 5.11 | 4.75 | 4.64 | 4.79 | 5.56 | 4.11 |
| cultivation for | | | | | | | | | | | | |
| 70.79 h (g/L) | | | | | | | | | | | | |

### Dry Biomass Concentration (DBM)

TABLE 52
Overview of the dry biomass concentrations for the six
replicates with medium 5 and medium 6 after 70.79 h.

| | Condition (% by wt. of sugars based on total sugar) | | | | | | | | | | | |
|---|---|---|---|---|---|---|---|---|---|---|---|---|
| | Medium 5 (50% by wt. of D-glucose, | | | | | | Medium 6 (39% by wt. of D-glucose, | | | | | |
| | 50% by wt. of D-xylose) | | | | | | 61% by wt. of D-xylose) | | | | | |
| | Position in MTP | | | | | | | | | | | |
| | D1 | D2 | D3 | D4 | D5 | D6 | D7 | D8 | E1 | E2 | E3 | E4 |
| DBM after | 5.33 | 4.43 | 4.01 | 4.27 | 4.19 | 4.73 | 3.88 | 2.09 | 3.52 | 2.63 | 3.57 | 3.19 |
| cultivation for | | | | | | | | | | | | |
| 70.79 h (g/L) | | | | | | | | | | | | |

### Dry Biomass Concentration (DBM)

TABLE 53
Overview of the dry biomass concentrations for the six
replicates with medium 7 and medium 8 after 70.79 h.

| | Condition (% by wt. of sugars based on total sugar) | | | | | | | | | | | |
|---|---|---|---|---|---|---|---|---|---|---|---|---|
| | Medium 7 (27% by wt. of D-glucose, | | | | | | Medium 8 (14% by wt. of D-glucose, | | | | | |
| | 73% by wt. of D-xylose) | | | | | | 86% by wt. of D-xylose) | | | | | |
| | Position in MTP | | | | | | | | | | | |
| | E5 | E6 | E7 | E8 | F1 | F2 | F3 | F4 | F5 | F6 | F7 | F8 |
| DBM after | 2.68 | 2.37 | 2.37 | 2.29 | 2.84 | 3.68 | #NV | 1.49 | 2.63 | 1.17 | 1.56 | 1.49 |
| cultivation for | | | | | | | | | | | | |
| 70.79 h (g/L) | | | | | | | | | | | | |

Ortho-Aminobenzoic Acid Concentration (oAB)

TABLE 54
Overview of the ortho-aminobenzoic acid concentration for the
six replicates with medium 1 and medium 2 after 70.79 h.

| | Condition (% by wt. of sugars based on total sugar) | | | | | | | | | | | |
|---|---|---|---|---|---|---|---|---|---|---|---|---|
| | Medium 1 (100% by wt. of D-glucose, | | | | | | Medium 2 (86% by wt. of D-glucose, | | | | | |
| | 0% by wt. of D-xylose) | | | | | | 14% by wt. of D-xylose) | | | | | |
| | Position in MTP | | | | | | | | | | | |
| | A1 | A2 | A3 | A4 | A5 | A6 | A7 | A8 | B1 | B2 | B3 | B4 |
| oAB after | 2.258 | 2.295 | 2.341 | 2.245 | 2.345 | 2.322 | 2.464 | 2.450 | 2.427 | 2.409 | 2.450 | 2.432 |
| cultivation | | | | | | | | | | | | |
| for 70.79 h | | | | | | | | | | | | |
| (g/L) | | | | | | | | | | | | |

Ortho-Aminobenzoic Acid Concentration (oAB)

TABLE 55
Overview of the ortho-aminobenzoic acid concentration for the
six replicates with medium 3 and medium 4 after 70.79 h.

| | Condition (% by wt. of sugars based on total sugar) | | | | | | | | | | | |
|---|---|---|---|---|---|---|---|---|---|---|---|---|
| | Medium 3 (76% by wt. of D-glucose, | | | | | | Medium 4 (63% by wt. of D-glucose, | | | | | |
| | 24% by wt. of D-xylose) | | | | | | 37% by wt. of D-xylose) | | | | | |
| | Position in MTP | | | | | | | | | | | |
| | B5 | B6 | B7 | B8 | C1 | C2 | C3 | C4 | C5 | C6 | C7 | C8 |
| oAB after | 2.425 | 2.469 | 2.430 | 2.448 | 2.441 | 2.400 | 2.341 | 2.336 | 2.343 | 2.315 | 2.357 | 2.345 |
| cultivation for | | | | | | | | | | | | |
| 70.79 h (g/L) | | | | | | | | | | | | |

Ortho-Aminobenzoic Acid Concentration (oAB)

TABLE 56
Overview of the ortho-aminobenzoic acid concentration for the
six replicates with medium 5 and medium 6 after 70.79 h.

| | Condition (% by wt. of sugars based on total sugar) | | | | | | | | | | | |
|---|---|---|---|---|---|---|---|---|---|---|---|---|
| | Medium 5 (50% by wt. of D-glucose, | | | | | | Medium 6 (39% by wt. of D-glucose, | | | | | |
| | 50% by wt. of D-xylose) | | | | | | 61% by wt. of D-xylose) | | | | | |
| | Position in MTP | | | | | | | | | | | |
| | D1 | D2 | D3 | D4 | D5 | D6 | D7 | D8 | E1 | E2 | E3 | E4 |
| oAB after | 2.119 | 2.103 | 2.098 | 2.085 | 2.082 | 2.137 | 1.783 | 1.758 | 1.778 | 1.758 | 1.717 | 1.719 |
| cultivation | | | | | | | | | | | | |
| for 70.79 h | | | | | | | | | | | | |
| (g/L) | | | | | | | | | | | | |

Ortho-Aminobenzoic Acid Concentration (oAB)

TABLE 57
Overview of the ortho-aminobenzoic acid concentration for the
six replicates with medium 7 and medium 8 after 70.79 h.

| | Condition (% by wt. of sugars based on total sugar) | | | | | | | | | | | |
|---|---|---|---|---|---|---|---|---|---|---|---|---|
| | Medium 7 (27% by wt. of D-glucose, | | | | | | Medium 8 (14% by wt. of D-glucose, | | | | | |
| | 73% by wt. of D-xylose) | | | | | | 86% by wt. of D-xylose) | | | | | |
| | Position in MTP | | | | | | | | | | | |
| | E5 | E6 | E7 | E8 | F1 | F2 | F3 | F4 | F5 | F6 | F7 | F8 |
| oAB after | 1.305 | 1.323 | 1.327 | 1.320 | 1.375 | 1.397 | 0.876 | 0.790 | 0.821 | 0.801 | 0.771 | 0.773 |
| cultivation | | | | | | | | | | | | |
| for 70.79 h | | | | | | | | | | | | |
| (g/L) | | | | | | | | | | | | |

### Final Product Yield

TABLE 58
Overview of the ortho-aminobenzoic acid yields after 70.79 h for six replicates with medium
1 and medium 2. These are reported as the mass-based (g_{oAB}/g_{C source}) and molar amount-based quotient
(mol_{oAB}/mol_{C source}), including firstly the metabolized carbon source and secondly the
carbon source included in the initial charge of medium in the calculation.

| | Condition (% by wt. of sugars based on total sugar) | | | | | | | | | | | |
|---|---|---|---|---|---|---|---|---|---|---|---|---|
| | Medium 1 (100% by wt. of D-glucose, | | | | | | Medium 2 (86% by wt. of D-glucose, | | | | | |
| | 0% by wt. of D-xylose) | | | | | | 14% by wt. of D-xylose) | | | | | |
| | Position in MTP | | | | | | | | | | | |
| | A1 | A2 | A3 | A4 | A5 | A6 | A7 | A8 | B1 | B2 | B3 | B4 |
| Yield after | 0.107 | 0.109 | 0.111 | 0.107 | 0.111 | 0.110 | 0.116 | 0.115 | 0.114 | 0.113 | 0.115 | 0.115 |
| cultivation | | | | | | | | | | | | |
| for 70.79 h | | | | | | | | | | | | |
| (g_{oAB}/ | | | | | | | | | | | | |
| g_{C source consumed}) | | | | | | | | | | | | |
| Yield after | 0.107 | 0.109 | 0.111 | 0.107 | 0.111 | 0.110 | 0.116 | 0.115 | 0.114 | 0.113 | 0.115 | 0.114 |
| cultivation | | | | | | | | | | | | |
| for 70.79 h | | | | | | | | | | | | |
| (go_{AB}/ | | | | | | | | | | | | |
| g_{C source initially charged}) | | | | | | | | | | | | |
| Yield after | 0.141 | 0.143 | 0.146 | 0.140 | 0.146 | 0.145 | 0.148 | 0.147 | 0.146 | 0.145 | 0.147 | 0.148 |
| cultivation | | | | | | | | | | | | |
| for 70.79 h | | | | | | | | | | | | |
| (mol_{oAB}/ | | | | | | | | | | | | |
| mol_{C source consumed}) | | | | | | | | | | | | |
| Yield after | 0.141 | 0.143 | 0.146 | 0.140 | 0.146 | 0.145 | 0.148 | 0.147 | 0.146 | 0.145 | 0.147 | 0.146 |
| cultivation | | | | | | | | | | | | |
| for 70.79 h | | | | | | | | | | | | |
| (mol_{oAB}/ | | | | | | | | | | | | |
| mol_{C source initially charged}) | | | | | | | | | | | | |

### Final Product Yield

TABLE 59
Overview of the ortho-aminobenzoic acid yields after 70.79 h for six replicates with medium
3 and medium 4. These are reported as the mass-based (g_{oAB}/g_{C source}) and molar amount-based quotient
(mol_{oAB}/mol_{C source}), including firstly the metabolized carbon source and secondly the
carbon source included in the initial charge of medium in the calculation.

| | Condition (% by wt. of sugars based on total sugar) | | | | | | | | | | | |
|---|---|---|---|---|---|---|---|---|---|---|---|---|
| | Medium 3 (76% by wt. of D-glucose, | | | | | | Medium 4 (63% by wt. of D-glucose, | | | | | |
| | 24% by wt. of D-xylose) | | | | | | 37% by wt. of D-xylose) | | | | | |
| | Position in MTP | | | | | | | | | | | |
| | B5 | B6 | B7 | B8 | C1 | C2 | C3 | C4 | C5 | C6 | C7 | C8 |
| Yield after | 0.120 | 0.123 | 0.121 | 0.122 | 0.121 | 0.121 | 0.124 | 0.123 | 0.127 | 0.125 | 0.127 | 0.129 |
| cultivation | | | | | | | | | | | | |
| for 70.79 h | | | | | | | | | | | | |
| (g_{oAB}/ | | | | | | | | | | | | |
| g_{C source consumed}) | | | | | | | | | | | | |
| Yield after | 0.118 | 0.120 | 0.118 | 0.119 | 0.118 | 0.116 | 0.113 | 0.113 | 0.114 | 0.112 | 0.114 | 0.114 |
| cultivation | | | | | | | | | | | | |
| for 70.79 h | | | | | | | | | | | | |
| (go_{AB}/ | | | | | | | | | | | | |
| g_{C source initially charged}) | | | | | | | | | | | | |
| Yield after | 0.151 | 0.155 | 0.152 | 0.153 | 0.152 | 0.152 | 0.154 | 0.153 | 0.157 | 0.156 | 0.158 | 0.160 |
| cultivation | | | | | | | | | | | | |
| for 70.79 h | | | | | | | | | | | | |
| (mol_{oAB}/ | | | | | | | | | | | | |
| mol_{C source consumed}) | | | | | | | | | | | | |
| Yield after | 0.147 | 0.150 | 0.148 | 0.149 | 0.148 | 0.146 | 0.139 | 0.139 | 0.139 | 0.137 | 0.140 | 0.139 |
| cultivation | | | | | | | | | | | | |
| for 70.79 h | | | | | | | | | | | | |
| (mol_{oAB}/ | | | | | | | | | | | | |
| mol_{C source initially charged}) | | | | | | | | | | | | |

### Final Product Yield

TABLE 60
Overview of the ortho-aminobenzoic acid yields after 70.79 h for six replicates with medium
5 and medium 6. These are reported as the mass-based (g_{oAB}/g_{C source}) and molar amount-based quotient
(mol_{oAB}/mol_{C source}), including firstly the metabolized carbon source and secondly the
carbon source included in the initial charge of medium in the calculation.

| | Condition (% by wt. of sugars based on total sugar) | | | | | | | | | | | |
|---|---|---|---|---|---|---|---|---|---|---|---|---|
| | Medium 5 (50% by wt. of D-glucose, | | | | | | Medium 6 (39% by wt. of D-glucose, | | | | | |
| | 50% by wt. of D-xylose) | | | | | | 61% by wt. of D-xylose) | | | | | |
| | Position in MTP | | | | | | | | | | | |
| | D1 | D2 | D3 | D4 | D5 | D6 | D7 | D8 | E1 | E2 | E3 | E4 |
| Yield after | 0.129 | 0.129 | 0.128 | 0.126 | 0.128 | 0.132 | 0.139 | 0.138 | 0.139 | 0.139 | 0.136 | 0.140 |
| cultivation | | | | | | | | | | | | |
| for 70.79 h | | | | | | | | | | | | |
| (g_{oAB}/ | | | | | | | | | | | | |
| g_{C source consumed}) | | | | | | | | | | | | |
| Yield after | 0.102 | 0.101 | 0.101 | 0.100 | 0.100 | 0.103 | 0.089 | 0.087 | 0.088 | 0.087 | 0.085 | 0.086 |
| cultivation | | | | | | | | | | | | |
| for 70.79 h | | | | | | | | | | | | |
| (g_{oAB}/ | | | | | | | | | | | | |
| g_{C source initially charged}) | | | | | | | | | | | | |
| Yield after | 0.158 | 0.158 | 0.157 | 0.154 | 0.157 | 0.162 | 0.169 | 0.168 | 0.170 | 0.169 | 0.167 | 0.172 |
| cultivation | | | | | | | | | | | | |
| for 70.79 h | | | | | | | | | | | | |
| (mol_{oAB}/ | | | | | | | | | | | | |
| mol_{C source consumed}) | | | | | | | | | | | | |
| Yield after | 0.121 | 0.120 | 0.120 | 0.119 | 0.119 | 0.122 | 0.104 | 0.102 | 0.104 | 0.102 | 0.100 | 0.100 |
| cultivation | | | | | | | | | | | | |
| for 70.79 h | | | | | | | | | | | | |
| (mol_{oAB}/ | | | | | | | | | | | | |
| mol_{C source initially charged}) | | | | | | | | | | | | |

### Final Product Yield

TABLE 61
Overview of the ortho-aminobenzoic acid yields after 70.79 h for six replicates with medium
7 and medium 8. These are reported as the mass-based (g_{oAB}/g_{C source}) and molar amount-based quotient
(mol_{oAB}/mol_{C source}), including firstly the metabolized carbon source and secondly the
carbon source included in the initial charge of medium in the calculation.

| | Condition (% by wt. of sugars based on total sugar) | | | | | | | | | | | |
|---|---|---|---|---|---|---|---|---|---|---|---|---|
| | Medium 7 (27% by wt. of D-glucose, | | | | | | Medium 8 (14% by wt. of D-glucose, | | | | | |
| | 73% by wt. of D-xylose) | | | | | | 86% by wt. of D-xylose) | | | | | |
| | Position in MTP | | | | | | | | | | | |
| | E5 | E6 | E7 | E8 | F1 | F2 | F3 | F4 | F5 | F6 | F7 | F8 |
| Yield after | 0.147 | 0.147 | 0.148 | 0.148 | 0.154 | 0.147 | 0.173 | 0.170 | 0.168 | 0.169 | 0.164 | 0.198 |
| cultivation | | | | | | | | | | | | |
| for 70.79 h | | | | | | | | | | | | |
| (g_{oAB}/ | | | | | | | | | | | | |
| g_{C source consumed}) | | | | | | | | | | | | |
| Yield after | 0.066 | 0.067 | 0.067 | 0.067 | 0.070 | 0.071 | 0.045 | 0.041 | 0.042 | 0.041 | 0.040 | 0.040 |
| cultivation | | | | | | | | | | | | |
| for 70.79 h | | | | | | | | | | | | |
| (g_{oAB}/ | | | | | | | | | | | | |
| g_{C source initially charged}) | | | | | | | | | | | | |
| Yield after | 0.178 | 0.178 | 0.179 | 0.180 | 0.187 | 0.178 | 0.208 | 0.205 | 0.203 | 0.204 | 0.198 | 0.244 |
| cultivation | | | | | | | | | | | | |
| for 70.79 h | | | | | | | | | | | | |
| (mol_{oAB}/ | | | | | | | | | | | | |
| mol_{C source consumed}) | | | | | | | | | | | | |
| Yield after | 0.076 | 0.077 | 0.077 | 0.077 | 0.080 | 0.081 | 0.051 | 0.046 | 0.047 | 0.046 | 0.045 | 0.045 |
| cultivation | | | | | | | | | | | | |
| for 70.79 h | | | | | | | | | | | | |
| (mol_{oAB}/ | | | | | | | | | | | | |
| mol_{C source initially charged}) | | | | | | | | | | | | |

### Averages of the Final Product Yields and Standard Deviations

TABLE 62
Overview of the averages and standard deviations of the ortho-aminobenzoic acid yields from six replicates in each case after 70.79
h. These are reported as the mass-based (g_{oAB}/g_{C source}) and molar amount-based quotient (mol_{oAB}/mol_{C source}), including firstly
the metabolized carbon source and secondly the carbon source included in the initial charge of medium in the calculation.

| | Medium No. (—) | | | | | | | |
|---|---|---|---|---|---|---|---|---|
| | Medium 1 | Medium 2 | Medium 3 | Medium 4 | Medium 5 | Medium 6 | Medium 7 | Medium 8 |
| | Condition (% by wt. of sugars based on total sugar) | | | | | | | |
| | 100% by | 86% by | 76% by | 63% by | 50% by | 39% by | 27% by | 14% by |
| | wt. of | wt. of | wt. of | wt. of | wt. of | wt. of | wt. of | wt. of |
| | D-glucose, | D-glucose, | D-glucose, | D-glucose, | D-glucose, | D-glucose, | D-glucose, | D-glucose, |
| | 0% by wt. | 14% by wt. | 24% by wt. | 37% by wt. | 50% by wt. | 61% by wt. | 73% by wt. | 86% by wt. |
| | of D-xylose | of D-xylose | of D-xylose | of D-xylose | of D-xylose | of D-xylose | of D-xylose | of D-xylose |
| Average yield | 0.109 ± | 0.115 ± | 0.121 ± | 0.126 ± | 0.129 ± | 0.139 ± | 0.149 ± | 0.174 ± |
| after | 0.002 | 0.001 | 0.001 | 0.002 | 0.002 | 0.001 | 0.003 | 0.012 |
| cultivation for | | | | | | | | |
| 70.79 h | | | | | | | | |
| (g_{oAB}/ | | | | | | | | |
| g_{C source consumed}) | | | | | | | | |
| Average yield | 0.109 ± | 0.115 ± | 0.118 ± | 0.113 ± | 0.101 ± | 0.087 ± | 0.068 ± | 0.042 ± |
| after | 0.002 | 0.001 | 0.001 | 0.001 | 0.001 | 0.001 | 0.002 | 0.002 |
| cultivation for | | | | | | | | |
| 70.79 h | | | | | | | | |
| (g_{oAB}/ | | | | | | | | |
| g_{C source initially charged}) | | | | | | | | |
| Average yield | 0.144 ± | 0.147 ± | 0.153 ± | 0.156 ± | 0.158 ± | 0.169 ± | 0.180 ± | 0.210 ± |
| after | 0.003 | 0.001 | 0.001 | 0.003 | 0.003 | 0.002 | 0.003 | 0.017 |
| cultivation for | | | | | | | | |
| 70.79 h | | | | | | | | |
| (mol_{oAB}/ | | | | | | | | |
| mol_{C source consumed}) | | | | | | | | |
| Average yield | 0.144 ± | 0.146 ± | 0.148 ± | 0.139 ± | 0.121 ± | 0.102 ± | 0.078 ± | 0.047 ± |
| after | 0.003 | 0.001 | 0.001 | 0.001 | 0.001 | 0.002 | 0.002 | 0.002 |
| cultivation for | | | | | | | | |
| 70.79 h | | | | | | | | |
| (mol_{oAB}/ | | | | | | | | |
| mol_{C source initially charged}) | | | | | | | | |

### SUMMARY

In the culturing described here of a xylose-metabolizing ortho-aminobenzoic acid producer based on C. glutamicum in microtiter plate format, the effect of various mixing ratios of glucose and xylose on the yield of ortho-aminobenzoic acid was examined over an extended mixing range.

By the time the cultures had ended, the xylose was in most cases still not yet fully consumed, and therefore a distinction was made in respect of the final product yield between the “substrate yield” based on the mass or molar amount of substrate consumed (g_{oAB}/g_{C source consumed} or mol_{oAB}/mol_{C source consumed}) and the “process yield” based on the mass or molar amount of the substrate initially charged (g_{oAB}/g_{C source initially charged} or mol_{oAB}/mol_{C source initially charged}).

It was found that metabolic yield rose with increasing proportion of xylose. In respect of process yield, a maximum of 0.153 g_{oAB}/g_{C source initially charged} or 0.148 mol_{oAB}/mol_{C source initially charged} was achieved with medium 3, in which the energy source and carbon source used was composed to an extent of 76% by weight of glucose and 24% by weight of xylose.

In this study, it was thus found that, surprisingly, the combination of D-glucose and D-xylose in mixtures having a glucose content of 86% to 14% by weight and a xylose content between 14% and 86% by weight leads to more efficient ortho-aminobenzoic acid product formation than the pure use of D-glucose as carbon source and energy source for production thereof.

### Example 3

In this example, it was examined whether the use of a longer (SEQ ID NO. 13) or shorter (SEQ ID NO. 12) variant of xylose isomerase xylA (xcc1758) from Xanthomonas campestris pv. campestris leads to the same results. For this purpose, variants of the above-describe strain were used, which differ solely in the variants of xylose isomerase present. The strain containing the extended variant of the enzyme as also used in examples 1 and 2 is referred to hereinafter as “Gizmo”, and the strain containing the shorter variant instead as “Geronimo”.

### Media

The media used in this example 3 can be found in tables 3 to 12 or tables 29 to 38 and table 63.

TABLE 63
Overview of production of SY medium. The medium
is made up with ddH₂O and autoclaved.

| | | Addition before/ |
|---|---|---|
| Medium | Composition | after the autoclave |
| SY medium | 16 g/L soy peptone | Before autoclaving |
| | 5 g/L sodium chloride | Before autoclaving |
| | 10 g/L yeast extract | Before autoclaving |
| | pH with KOH to pH = 7.2 | Before autoclaving |
| | Glucose (final | After autoclaving |
| | concentration: 16 g/L) | |

The cultivation parameters corresponded to those from table 27, except that the cultivation time in example 3 was 70.95 h.

### Instruments Used

Apart from the measurement of dry biomass, in example 3, the parameters from table 39 were analyzed with the aid of the instruments from table 39, and the instruments in table 40 were used for the culturing.

### Procedure

A starter culture was generated by taking cell mass from dormant forms in glycerol of the microbial cultures used for the culturing, and these were used to inoculate a BHI agar plate. The BHI agar plate was incubated at 30° C. for 48 h and stored if necessary at 4° C. until further use.

The agar plate culture set up in this way was used to inoculate an SY liquid culture. For this purpose, a little cell mass was taken from the BHI agar plate culture and 4 mL of liquid SY medium was inoculated in a round-bottom tube. The liquid culture was incubated at 30° C. and 200 rpm for about 7.5 h (preparatory culture I).

For the second preparatory culture, CGXII preparatory culture medium with 15 g/L D-glucose and 5 g/L D-xylose was produced, and 24 mL was transferred into each of two 500 mL Erlenmeyer flasks. 1 ml of the liquid SY preparatory culture was added to each of the two batches, and these two shaken flask preparatory cultures were incubated at 200 rpm and 30° C. for 17 h.

On completion of incubation of the second preparatory cultures in the shaken flask, the optical density of the cultures, which had a mixture of glucose and xylose available as the carbon source, was measured. Depending on the cell density achieved, a proportion of these second preparatory cultures was taken, the cells were pelletized by centrifugation and washed in sterile phosphate buffer (singly concentrated), and resuspended in appropriate main culture medium.

TABLE 64
Measurement of optical density (OD₆₀₀) in double determination
on completion of incubation of the second preparatory cultures
in the shaken flask. The averages of the optical density
measured were used to calculate what volume of the second
preparatory culture is needed in each case to inoculate 25 mL
of main culture of C. glutamicum Gizmo and C. glutamicum
Geronimo with an initial OD₆₀₀ of 1.

| Preparatory | OD₆₀₀ | OD₆₀₀ | Average | Volume |
|---|---|---|---|---|
| culture II | (—) | (—) | OD ₆₀₀ (—) | required (mL) |
| C. glutamicum Gizmo | 17 | 16 | 16.5 | 1.52 |
| C. glutamicum | 16 | 17 | 16.5 | 1.52 |
| Geronimo | | | | |

The main cultures of C. glutamicum Gizmo and C. glutamicum Geronimo that had been produced in this way had an identical initial sugar ratio of 76% by weight of D-glucose and 24% by weight of D-xylose based on total sugar. Aliquots of these main cultures were pipetted into different positions of a culture plate, and the culture plate was sealed with a film and transferred into the BioLector pro.

### Results

### D-Xylose Concentration

TABLE 65
Overview of the D-xylose concentrations at the start and at the end of culturing
for three replicates in each case of C. glutamicum Gizmo and C. glutamicum
Geronimo. The medium contained initial sugar contents of 76% by weight of D-glucose
and 24% by weight of D-xylose based on total sugar. The initial D-xylose concentration
was determined in uninoculated main culture medium.

| | Name of culture, condition (% by wt. of sugars based on total sugar) | | | | | |
|---|---|---|---|---|---|---|
| | C. glutamicum Gizmo, | | | C. glutamicum Geronimo, | | |
| | 76% by wt. of D-glucose, | | | 76% by wt. of D-glucose, | | |
| | 24% by wt. of D-xylose | | | 24% by wt. of D-xylose | | |
| | Replicate (—) | | | | | |
| | 1 | 2 | 3 | 1 | 2 | 3 |
| Culture time | D-Xylose | D-Xylose | D-Xylose | D-Xylose | D-Xylose | D-Xylose |
| (h) | (g/L) | (g/L) | (g/L) | (g/L) | (g/L) | (g/L) |
| 0.00 | 4.93 | 4.93 | 4.93 | 4.93 | 4.93 | 4.93 |
| 70.95 | 0.64 | 0.70 | 0.81 | 0.34 | 0.18 | 0.19 |

### D-Glucose Concentration

TABLE 66
Overview of the D-glucose concentrations at the start and at the end of culturing
for three replicates in each case of C. glutamicum Gizmo and C. glutamicum
Geronimo. The medium contained initial sugar contents of 76% by weight of D-glucose
and 24% by weight of D-xylose based on total sugar. The initial D-glucose concentration
was determined in uninoculated main culture medium.

| | Name of culture, condition (% by wt. of sugars based on total sugar) | | | | | |
|---|---|---|---|---|---|---|
| | C. glutamicum Gizmo, | | | C. glutamicum Geronimo, | | |
| | 76% by wt. of D-glucose, | | | 76% by wt. of D-glucose, | | |
| | 24% by wt. of D-xylose | | | 24% by wt. of D-xylose | | |
| | Replicate (—) | | | | | |
| | 1 | 2 | 3 | 1 | 2 | 3 |
| Culture time | D-Glucose | D-Glucose | D-Glucose | D-Glucose | D-Glucose | D-Glucose |
| (h) | (g/L) | (g/L) | (g/L) | (g/L) | (g/L) | (g/L) |
| 0.00 | 15.63 | 15.63 | 15.63 | 15.63 | 15.63 | 15.63 |
| 70.95 | 0.00 | 0.00 | 0.00 | 0.00 | 0.00 | 0.00 |

Ortho-Aminobenzoic Acid Concentration (oAB)

TABLE 67
Overview of the ortho-aminobenzoic acid concentrations (oAB) at the start and at
the end of culturing for three replicates in each case of C. glutamicum Gizmo and
C. glutamicum Geronimo. The medium contained initial sugar contents of 76% by
weight of D-glucose and 24% by weight of D-xylose based on total sugar.

| | Name of culture, condition (% by wt. of sugars based on total sugar) | | | | | |
|---|---|---|---|---|---|---|
| | C. glutamicum Gizmo, | | | C. glutamicum Geronimo, | | |
| | 76% by wt. of D-glucose, | | | 76% by wt. of D-glucose, | | |
| | 24% by wt. of D-xylose | | | 24% by wt. of D-xylose | | |
| | Replicate (—) | | | | | |
| | 1 | 2 | 3 | 1 | 2 | 3 |
| Culture time | oAB | oAB | oAB | oAB | oAB | oAB |
| (h) | (g/L) | (g/L) | (g/L) | (g/L) | (g/L) | (g/L) |
| 0.00 | 0 | 0 | 0 | 0 | 0 | 0 |
| 70.95 | 2.46 | 2.49 | 2.48 | 2.45 | 2.42 | 2.43 |

### Final Product Yield

TABLE 68
Overview of the ortho-aminobenzoic acid yields after 70.95 h for three replicates
in each case of C. glutamicum Gizmo and C. glutamicum Geronimo in medium
with initial sugar contents of 74% by weight of D-glucose and 26% by weight of D-
xylose based on total sugar. These are reported as the mass-based (g_{oAB}/g_{C source}) and
molar amount-based quotient (mol_{oAB}/mol_{C source}), including firstly the metabolized
carbon source and secondly the carbon source included in the initial charge
of medium in the calculation.

| | Name of culture, condition | | | | | |
|---|---|---|---|---|---|---|
| | (% by wt. of sugars based on total sugar) | | | | | |
| | C. glutamicum Gizmo, | | | C. glutamicum Geronimo, | | |
| | 76% by wt. of D-glucose, | | | 76% by wt. of D-glucose, | | |
| | 24% by wt. of D-xylose | | | 24% by wt. of D-xylose | | |
| | Replicate (—) | | | | | |
| | 1 | 2 | 3 | 1 | 2 | 3 |
| Yield after | 0.124 | 0.125 | 0.126 | 0.121 | 0.119 | 0.119 |
| cultivation for | | | | | | |
| 70.95 h (g_{oAB}/ | | | | | | |
| g_{C source consumed}) | | | | | | |
| Yield after | 0.120 | 0.121 | 0.121 | 0.119 | 0.118 | 0.118 |
| cultivation for | | | | | | |
| 70.95 h (g_{oAB}/ | | | | | | |
| g_{C source initially charged}) | | | | | | |
| Yield after | 0.156 | 0.158 | 0.158 | 0.152 | 0.149 | 0.150 |
| cultivation for | | | | | | |
| 70.95 h (mol_{oAB}/ | | | | | | |
| mol_{C source consumed}) | | | | | | |
| Yield after | 0.150 | 0.152 | 0.151 | 0.149 | 0.147 | 0.148 |
| cultivation for | | | | | | |
| 70.95 h (mol_{oAB}/ | | | | | | |
| mol_{C source initially charged}) | | | | | | |

### Averages of the Final Product Yields and Standard Deviations

TABLE 69
Overview of the averages and standard deviations of the ortho-aminobenzoic acid
yields from three replicates in each case of C. glutamicum Gizmo and C. glutamicum
Geronimo in medium with initial sugar contents of 74% by weight of D-glucose and
26% by weight of D-xylose based on total sugar after 70.95 h. These are reported
as the mass-based (goAB/gc source) and molar amount-based quotient (moloAB/molc
source), including firstly the metabolized carbon source and secondly the carbon
source included in the initial charge of medium in the calculation.

| | C. glutamicum | C. glutamicum |
|---|---|---|
| Name of culture, | Gizmo, | Geronimo, |
| condition (% by wt. of | 76% by wt. of D- | 76% by wt. of D- |
| sugars based on total | glucose, 24% by wt. | glucose, 24% by wt. |
| sugar) | of D-xylose | of D-xylose |
| Average yield after | 0.125 ± 0.001 | 0.120 ± 0.001 |
| cultivation for 70.95 h | | |
| (goAB/gC source consumed) | | |
| Average yield after | 0.120 ± 0.001 | 0.118 ± 0.001 |
| cultivation for 70.95 h | | |
| (goAB/ gC source initially charged) | | |
| Average yield after | 0.157 ± 0.001 | 0.150 ± 0.002 |
| cultivation for 70.95 h | | |
| (moloAB/molc source consumed) | | |
| Average yield after | 0.151 ± 0.001 | 0.148 ± 0.000 |
| cultivation for 70.95 h | | |
| (moloAB/molc source initially | | |
| charged) | | |

### SUMMARY

In the culturing described here of two xylose-metabolizing ortho-aminobenzoic acid producers based on C. glutamicum in microtiter plate format, the effect of various xylose isomerase variants on the yield of ortho-aminobenzoic acid was examined.

Example 3 shows that the use of the two different variants of xylose isomerase does not lead to relevant differences in the performance of the strains. Both variants are thus equivalent in the context of the present invention.

1. A method of producing ortho-aminobenzoic acid (oAB) comprising culturing one or more cells of the Corynebacterium genus that are able to convert glucose and xylose to oAB in a culture medium containing a mixture of glucose and xylose with a glucose content between 5% by weight and 86% by weight and a xylose content between 95% by weight and 14% by weight,
wherein the proportions of glucose and xylose add up to 100%;
wherein oAB is produced; and
wherein said cells differ from the wild type at least in the following features:
(i) reduced expression of anthranilate phosphoribosyltransferase compared to the wild type, but where there must be residual activity;
(ii) elevated activity of shikimate kinase;
(iii) elevated activity of 3-phosphoshikimate 1-carboxyvinyltransferase and chorismate synthase;
(iv) presence of a feedback-resistant 3-deoxyarabinoheptulosanate-7-phosphate synthase; and
(v) elevated activity of xylose isomerase and of xylulokinase.

2. The method as claimed in claim 1, wherein the mixture of glucose and xylose has a glucose content between 16% by weight and 86% by weight, and xylose contributes the proportion lacking from 100% by weight.

3. The method as claimed in claim 2, wherein the carbon yield is at least 0.138 mol of carbon in the form of oAB per mole of carbon consumed in glucose and xylose.

4. A cell of the Corynebacterium genus which is suitable for release of oAB and differs from the wild type at least in the following features:
(i) reduced expression of anthranilate phosphoribosyltransferase compared to the wild type, but where there must be residual activity;
(ii) elevated activity of shikimate kinase;
(iii) elevated activity of 3-phosphoshikimate 1-carboxyvinyltransferase and chorismate synthase;
(iv) presence of a feedback-resistant 3-deoxyarabinoheptulosanate-7-phosphate synthase; and
(v) elevated activity of xylose isomerase and of xylulokinase.

5. A method of producing OaB comprising microbial fermentation of strain of the Corynebacterium genus as claimed in claim 4 with a mixture of glucose and xylose as energy source and carbon source.

6. The method as claimed in claim 5, wherein the mixture of glucose and xylose has a glucose content between 5% by weight and 86% by weight and a xylose content between 95% by weight and 14% by weight, and wherein the proportions of glucose and xylose add up to 100%.

7. A composition comprising
a) microbial cells of the Corynebacterium genus as claimed in claim 4;
b) a mixture of glucose and xylose having a glucose content between 5% by weight and 86% by weight and a xylose content between 95% by weight and 14% by weight, where the proportions of glucose and xylose add up to 100%; and
c) at least one nitrogen source, at least one phosphorus source, at least one sulfur source and trace elements.

8. The composition as claimed in claim 7, additionally comprising oAB.

9. A method of producing oAB comprising microbial fermentation of a culture medium comprising a mixture of glucose and xylose having a glucose content between 5% by weight and 86% by weight and a xylose content between 95% by weight and 14% by weight, where the proportions of glucose and xylose add up to 100%.
